SEQUENCE LISTING (20 sequences)

SEQ ID NO: 1 - protein, 243 aa, Homo sapiens
  misc_feature (1)..(243): Human IL-27 subunit p28
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 2 - protein, 229 aa, Homo sapiens
  misc_feature (1)..(229): Human IL-27 subunit EBI3
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 3 - protein, 234 aa, Mouse
  misc_feature (1)..(234): Mouse IL-27 subunit p28
MGQVTGDLGWRLSLLLLPLLLVQAGSWGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 4 - protein, 228 aa, Mouse
  misc_feature (1)..(228): Mouse IL-27 subunit EBI3
MSKLLFLSLALWASRSPGYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 5 - protein, 636 aa, Homo sapiens
  misc_feature (1)..(636): Human IL-27 receptor, alpha subunit (WSX-1), with signal peptide
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 6 - protein, 918 aa, Homo sapiens
  misc_feature (1)..(918): Human gp130, with signal peptide
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 7 - protein, 623 aa, Mouse
  misc_feature (1)..(623): Mouse IL-27 receptor, alpha subunit (WSX-1), with signal peptide
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRWKALPWFLSLWGLLLMGCGLSLASTRCLQARCLHWRHKLLPQWIWERVPDPANSNSGQPYIKEVSLPQPPKDGPILEVEEVELQPVVESPKASAPIYSGYEKHFLPTPEELGLLV

SEQ ID NO: 8 - protein, 917 aa, Mouse
  misc_feature (1)..(917): Mouse gp130
MSAPRIWLAQALLFFLTTESIGQLLEPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDRPSRPPSFWYKTNPSHGQEYRSVRLIWKALPLSEANGKILDYEVILTQSKSVSQTYTVTGTELTVNLTNDRYVASLAARNKVGKSAAAVLTIPSPHVTAAYSVVNLKAFPKDNLLWVEWTPPPKPVSKYILEWCVLSENAPCVEDWQQEDATVNRTHLRGRLLESKCYQITVTPVFATGPGGSESLKAYLKQAAPARGPTVRTKKVGKNEAVLAWDQIPVDDQNGFIRNYSISYRTSVGKEMVVHVDSSHTEYTLSSLSSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANNKKPCPDDLKSVDLFKKEKVSTEGHSSGIGGSSCMSSSRPSISSNEENESAQSTASTVQYSTVVHSGYRHQVPSVQVFSRSESTQPLLDSEERPEDLQLVDSVDGGDEILPRQPYFKQNCSQPEACPEISHFERSNQVLSGNEEDFVRLKQQQVSDHISQPYGSEQRRLFQEGSTADALGTGADGQMERFESVGMETTIDEEIPKSYLPQTVRQGGYMPQ

SEQ ID NO: 9 - protein, 482 aa, Homo sapiens
  misc_feature (1)..(482): Human WSX-1 extracellular domain (ECD), to aa 482, with signal peptide
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPW

SEQ ID NO: 10 - protein, 450 aa, Homo sapiens
  misc_feature (1)..(450): Human WSX-1 ECD, to aa 482, without signal peptide
QGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPW

SEQ ID NO: 11 - protein, 232 aa, Homo sapiens
  misc_feature (1)..(232): Fc C237S
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 228 aa, Homo sapiens
  misc_feature (1)..(228): Exemplary Fc #1
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 229 aa, Homo sapiens
  misc_feature (1)..(229): Exemplary Fc #2
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 604 aa, Homo sapiens
  misc_feature (1)..(604): Human WSX-1, without signal peptide
QGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 15 - protein, 599 aa, Mouse
  misc_feature (1)..(599): Mouse WSX-1, without signal peptide
TRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRWKALPWFLSLWGLLLMGCGLSLASTRCLQARCLHWRHKLLPQWIWERVPDPANSNSGQPYIKEVSLPQPPKDGPILEVEEVELQPVVESPKASAPIYSGYEKHFLPTPEELGLLV

SEQ ID NO: 16 - protein, 510 aa, Mouse
  misc_feature (1)..(510): Mouse WSX-1 extracellular domain (ECD), to aa 510, with signal peptide
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRWK

SEQ ID NO: 17 - protein, 486 aa, Mouse
  misc_feature (1)..(486): Mouse WSX-1 ECD, to aa 510, without signal peptide
TRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRWK

SEQ ID NO: 18 - protein, 896 aa, Homo sapiens
  misc_feature (1)..(896): Human gp130, without signal peptide
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 19 - protein, 516 aa, Homo sapiens
  misc_feature (1)..(516): Human WSX-1 ECD, to aa 516, with signal peptide
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWK

SEQ ID NO: 20 - protein, 484 aa, Homo sapiens
  misc_feature (1)..(484): Human WSX-1 ECD, to aa 516, without signal peptide
QGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWK

(19) United States
(12) Patent Application Publication
(10) Pub. No.: US 2013/0189262 A1
(43) Pub. Date: Jul. 25, 2013

(54) IL-27 Antagonists for Treating Inflammatory Diseases

(71) Applicants: Brian Wong, Los Altos, CA (US); Jennifa Gosling, San Francisco, CA (US)

(72) Inventors: Brian Wong, Los Altos, CA (US); Jennifa Gosling, San Francisco, CA (US)

(73) Assignee: Five Prime Therapeutics, Inc., South San Francisco, CA (US)

(21) Appl. No.: 13/833,570

(22) Filed: Mar. 15, 2013

Related U.S. Application Data
(63) Continuation of application No. 13/350,122, filed on Jan. 13, 2012.
(60) Provisional application No. 61/432,921, filed on Jan. 14, 2011.

(51) Int. Cl. A61K 39/42; A61K 38/02; A61K 45/06; A61K 39/395

(52) U.S. Cl. CPC A61K 39/42; A61K 39/3955; A61K 38/02; A61K 45/06
USPC 424/135.1; 424/158.1; 424/172.1; 424/159.1; 514/1.7; 514/1.1; 424/133.1; 435/375

(57) ABSTRACT

Methods of treatment using IL-27 antagonists are provided. Such methods include, but are not limited to, methods of treating steroid-resistant conditions, such as asthma, chronic obstructive pulmonary disease (COPD), systemic lupus erythematosus (SLE), and inflammatory bowel disease. Such antagonists include, but are not limited to, antibodies that bind IL-27 and inhibit IL-27-mediated signaling (such as, for example, by blocking binding of IL-27 to its receptor); antibodies that bind the IL-27 receptor, alpha subunit, and inhibit IL-27-mediated signaling (such as, for example, by blocking binding of IL-27 to the receptor); and soluble forms of IL-27RA.

This application is a continuation of U.S. patent application Ser. No. 13/350,122, filed Jan. 13, 2012, which claims the benefit of U.S. Provisional Application No. 61/432,921, filed Jan. 14, 2011, each of which is incorporated by reference herein in its entirety for any purpose.

### TECHNICAL FIELD

Methods of treatment using IL-27 antagonists are provided. Such methods include, but are not limited to, methods of treating steroid-resistant conditions, such as steroid-resistant asthma, chronic obstructive pulmonary disease (COPD), steroid-resistant systemic lupus erythematosus (SLE), and steroid-resistant inflammatory bowel disease. Such antagonists include, but are not limited to, antibodies that bind IL-27 and inhibit IL-27-mediated signaling (such as, for example, by blocking binding of IL-27 to its receptor); antibodies that bind the IL-27 receptor, alpha subunit, and inhibit IL-27-mediated signaling (such as, for example, by blocking binding of IL-27 to the receptor); and soluble forms of IL-27RA.

### BACKGROUND

Asthma and chronic obstructive pulmonary disease (COPD) are the most common inflammatory diseases of the airways. Inflammation in the airway results in airway narrowing in both diseases, although the triggers for the inflammation vary. Asthma, particularly severe asthma, and COPD are often resistant to the most commonly prescribed therapies, such as steroids.

Other conditions commonly treated with steroids include systemic lupus erythematosus (SLE), and inflammatory bowel disease. Like asthma and COPD, each of those conditions may also be resistant to steroid therapy.

### SUMMARY

In some embodiments, methods of treating conditions comprising administering an IL-27 antagonist to a subject with the condition are provided, wherein the condition is selected from steroid-resistant asthma, Th2-low asthma, chronic obstructive pulmonary disease (COPD), steroid-resistant systemic lupus erythematosus (SLE), and steroid-resistant inflammatory bowel disease. In some embodiments, methods of treating airway inflammation comprising administering an IL-27 antagonist to a subject with airway inflammation are provided. In some embodiments, methods of treating steroid-resistant airway inflammation comprising administering an IL-27 antagonist to a subject with steroid-resistant airway inflammation are provided. In some embodiments, methods of treating airway hyperresponsiveness comprising administering an IL-27 antagonist to a subject with airway hyperresponsiveness are provided. In some embodiments, the airway hyperresponsiveness is steroid-resistant. In some embodiments, the condition is selected from steroid-resistant asthma, Th2-low asthma, and COPD.

In some embodiments, a condition has previously been characterized as having an elevated level of at least one protein selected from CXCL9, CXCL10, CXCL11, CD38, and WSX-1 in a subject's bronchial smooth muscle cells. In some embodiments, a condition has previously been characterized as having an elevated level of at least one protein selected from CXCL9, CXCL10, CD38, and WSX-1 in a subject's bronchial smooth muscle cells. In some embodiments, a condition has previously been characterized as having an elevated level of at least one protein selected from WSX-1, CXCL9, CXCL10, and CXCL11 in a subject's bronchial epithelial cells. In some embodiments, a condition has previously been characterized as having an elevated level of at least one protein selected from CXCL9, and CXCL10 in a subject's bronchial epithelial cells.

In some embodiments, a condition has previously been characterized as having an elevated level of at least one protein selected from IL-27 heterodimer, p28, TNF-α, and an interferon (such as IFN-α or IFN-γ) in a sample from a subject's lung. In some embodiments, the sample is selected from a bronchoalveolar lavage sample and a sputum sample (including, but not limited to, an induced sputum sample). a condition has previously been characterized as having an elevated level of at least one protein selected from IL-27 heterodimer, p28, TNF-α, and an interferon (such as IFN-α or IFN-γ) in at least one cell type from a subject's lung. In some embodiments, at least one cell type is a macrophage.

In some embodiments, methods of treating steroid-resistant airway inflammation are provided, wherein the method comprises administering an IL-27 antagonist to a subject with steroid-resistant airway inflammation.

In some embodiments, methods of reducing expression of at least one, at least two, at least three, at least four, or at least five genes selected from CXCL9, CXCL10, CXCL11, WSX-1, and CD38 in bronchial smooth muscle cells and/or bronchial epithelial cells are also provided, wherein the method comprises contacting the cells with an IL-27 antagonist. In some embodiments, methods of reducing expression of at least one, at least two, at least three, or at least four gene selected from CXCL9, CXCL10, WSX-1, and CD38 in bronchial smooth muscle cells and/or bronchial epithelial cells are also provided, wherein the method comprises contacting the cells with an IL-27 antagonist. In some embodiments, methods of increasing the steroid sensitivity of bronchial smooth muscle cells and/or bronchial epithelial cells are provided, wherein the method comprises contacting the cells with an IL-27 antagonist.

In some embodiments, the IL-27 antagonist is selected from an antibody that binds IL-27, an antibody that binds p28, an antibody that binds EBI3, an antibody that binds IL-27 receptor (IL-27R), an antibody that binds WSX-1, a WSX-1 extracellular domain (ECD), and a WSX-1 ECD fusion molecule. In some embodiments, the IL-27 antagonist is selected from an antibody that binds IL-27, an antibody that binds p28, and an antibody that binds EBI3. In some embodiments, the IL-27 antagonist is an antibody that binds p28. In some such embodiments, that antibody that binds p28 binds the IL-27 heterodimer. In some embodiments, an antibody that binds p28 and binds the IL-27 heterodimer does not bind to EBI3. In some embodiments, an antibody inhibits IL-27-mediated signaling. In some embodiments, the IL-27 antagonist is an antibody that binds WSX-1. In some embodiments, the antibody is selected from a chimeric antibody, a humanized antibody, and a human antibody. In some embodiments, the antibody is an antibody fragment. In some embodiments, the antibody fragment is selected from an Fv, a single-chain Fv (scFv), a Fab, a Fab′, and a (Fab′)₂.

In some embodiments, the IL-27 antagonist is a WSX-1 extracellular domain (ECD). In some embodiments, the IL-27 antagonist is a WSX-1 ECD fusion molecule. In some embodiments, the WSX-1 ECD fusion molecule comprises a WSX-1 ECD and at least one fusion partner. In some embodiments, at least one fusion partner is selected from an Fc, albumin, and polyethylene glycol. In some embodiments, at least one fusion partner is an Fc. In some embodiments, the at least one fusion partner is an Fc and polyethylene glycol. In some embodiments, at least one fusion partner is polyethylene glycol.

In some embodiments, a method of treating a condition is provided, wherein the method comprises administering an antibody that binds p28 and inhibits IL-27 mediated signaling to a subject with the condition, wherein the condition is selected from steroid-resistant asthma, Th2-low asthma, and chronic obstructive pulmonary disease (COPD). In some embodiments, the antibody binds p 28 and binds the IL-27 heterodimer, but does not bind EBI3.

In some embodiments, a method further comprises administering the subject at least one additional therapeutic selected from an anti-inflammatory agent and a bronchodilator. In some embodiments, the additional therapeutic is an anti-inflammatory agent. In some embodiments, the anti-inflammatory agent is selected from a steroid, a mast cell stabilizer, a leukotriene antagonist, omalizumab, roflumilast, and cilomilast. In some embodiments, the steroid is selected from prednisone, prednisolone, methylprednisone, fluticasone, budesonide, mometasone, triamcinolone, beclometasone, dexamethasone, and betamethasone; the mast cell stabilizer is selected from cromoglicic acid, nedocromil sodium; and the leukotriene antagonist is selected from montelukast, zafirlukast, and zileuton. In some embodiments, the additional therapeutic is a bronchodilator. In some embodiments, the bronchodilator is selected from a β₂ agonist, an anticholinergic, and theophylline. In some embodiments, the β₂ agonist is selected from albuterol, terbutaline, slameterol, and formoterol; and the anticholinergic is selected from ipratropium and tiotropium.

In some embodiments, an IL-27 antagonist restores steroid sensitivity in vitro in primary bronchial smooth muscle cells and/or primary bronchial epithelial cells contacted with TNF-α and IL-27.

Any embodiment described herein or any combination thereof applies to any and all IL-27 antagonists, including IL-27 antibodies, and methods and uses of the invention described herein.

### BRIEF DESCRIPTION OF THE FIGURES

FIG. 1 shows exemplary results of a screen to identify test substances that cause steroid-resistance in bronchial smooth muscle cells, as described in Example 1.

FIG. 2 shows exemplary results of two separate retests (open circles and closed circles) of test substances identified in the screen to identify test substances that cause steroid-resistance in bronchial smooth muscle cells, as described in Example 1.

FIG. 3 shows dose-dependent IL-27-induced steroid insensitivity in bronchial smooth muscle cells contacted with TNF-α and fluticasone, as described in Example 2.

FIG. 4 shows expression of CXCL10 in bronchial smooth muscle cells contacted with various combinations of factors, as described in Example 3.

FIG. 5 shows expression of CXCL10 in bronchial smooth muscle cells contacted with TNF-α, fluticasone, and various members of the IL-12 family of cytokines, as described in Example 4.

FIG. 6 shows expression levels of WSX-1 in various human tissues and cells, as described in Example 5.

FIG. 7 shows expression of WSX-1 in two different primary bronchial smooth muscle cell samples contacted with various factors, as described in Example 5.

FIG. 8 shows (A) induction of CXCL9 by TNF-α in primary human bronchial epithelial cells from a normal donor in the presence and absence of 25 nM fluticasone, and (B) induction of CXCL9 in primary human bronchial epithelial cells from a normal donor by IL-27 in the presence and absence of 25 nM fluticasone, as described in Example 6.

FIG. 9 shows induction of CXCL10 by IL-27 in primary human bronchial epithelial cells from a normal donor in the presence and absence of 25 nM fluticasone, as described in Example 6.

FIG. 10 shows (A) induction of CXCL9 and (B) induction of CXCL10 by IL-27 in primary human bronchial epithelial cells from a COPD patient in the presence and absence of 25 nM fluticasone; and (C) induction of CXCL9 and (D) induction of CXCL10 by IL-27 and TNF-α in primary human bronchial epithelial cells from a COPD patient in the presence and absence of 25 nM fluticasone, as described in Example 6.

FIG. 11 shows inhibition of IL-27-induced expression of CXCL10 by WSX-1 extracellular domain (ECD), as described in Example 7. All conditions except “no cytokine treatment” include 5 ng/ml TNF-α.

FIG. 12 shows inhibition of IL-27-induced expression of CXCL10 by a polyclonal antibody against IL-27, as described in Example 7.

### DETAILED DESCRIPTION

In a screen of over 4000 secreted and extracellular domain proteins to identify proteins involved in steroid resistance, IL-27 was found to induce a steroid-resistant state in bronchial smooth muscle cells when administered in combination with the pro-inflammatory cytokine TNF-α. The inventors discovered that bronchial smooth muscle cells and bronchial epithelial cells contacted with TNF-α and IL-27 show marked increases in expression of various inflammation marker genes, including IP-10 (CXCL10), MIG (CXCL9), and CD38. Further, while steroid treatment effectively down-regulates expression of genes induced by TNF-α alone, steroid treatment fails to down-regulate expression in the presence of TNF-α and IL-27. Addition of an IL-27 antagonist, such as a WSX-1 extracellular domain (ECD) or an antibody against IL-27, effectively inhibits IL-27 induced expression of CXCL10 in bronchial smooth muscle cells.

Th2-high asthma involves eosinophilic inflammation and responds to corticosteroids. Th2-low asthma, on the other hand, tends to be steroid-resistant. Further, the airway inflammation seen in COPD, which also tends to be steroid-resistant, is similar to that seen in severe asthma. Since existing asthma therapies are predominantly directed to Th2-high asthma, steroid-resistant Th2-low asthmatics and patients with COPD are left without effective therapy. The present invention provides IL-27 antagonists for treating steroid-resistant asthma and COPD. The present invention also provides IL-27 antagonists for treating other conditions, such as systemic lupus erythematosus (SLE) and inflammatory bowel disease.

The section headings used herein are for organizational purposes only and are not to be construed as limiting the subject matter described.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Exemplary techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (2nd ed., Cold Spring Harbor Laboratory Press, Cold Spring Harbor, N.Y. (1989)), among other places. In addition, exemplary techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Unless otherwise indicated, the term “include” has the same meaning as “include, but are not limited to,” the term “includes” has the same meaning as “includes, but is not limited to,” and the term “including” has the same meaning as “including, but not limited to.” Similarly, the term “such as” has the same meaning as the term “such as, but not limited to.” Also, terms such as “element” or “component” encompass both elements and components comprising one unit and elements and components that comprise more than one subunit unless specifically stated otherwise.

All references cited herein, including patent applications and publications, are incorporated by reference in their entirety.

As utilized in accordance with the present disclosure, the following terms, unless otherwise indicated, shall be understood to have the following meanings:

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like.

The term “IL-27” refers herein to a heterodimeric cytokine comprising the subunits p28 and EBI3. IL-27, as used herein, further refers to any native IL-27 from any vertebrate source, including mammals such as primates (e.g. humans) and rodents (e.g., mice and rats), unless otherwise indicated. The term encompasses full-length, unprocessed IL-27 as well as any form of IL-27 that results from processing in the cell or any fragment thereof. The term also encompasses naturally occurring variants of IL-27, e.g., splice variants or allelic variants. In some embodiments, IL-27 is a human IL-27 comprising a p28 (also referred to as IL-27A or IL-30) having the amino acid sequence of SEQ ID NO: 1 and an EBI3 (also referred to as IL-27B) having the amino acid sequence of SEQ ID NO: 2.

The terms “IL-27 receptor” and “IL-27R” refer herein to a heterodimeric receptor comprising IL-27 receptor, alpha subunit (referred to interchangeably as “IL-27RA,” “TCCR,” or “WSX-1”) and gp130. In some embodiments, IL-27 receptor is a human IL-27 receptor comprising a WSX-1 having the amino acid sequence of SEQ ID NO: 5 or SEQ ID NO: 14 and a gp130 having the amino acid sequence of SEQ ID NO: 6 or SEQ ID NO: 18.

The term “IL-27 activity” or “biological activity” of IL-27, as used herein, includes any biological effect of IL-27. In some embodiments, IL-27 activity includes the ability of IL-27 to interact or bind to a substrate or receptor. In some embodiments, the biological activity of IL-27 is the ability of IL-27 to stimulate STAT1 phosphorylation. In some embodiments, the overexpression of IL-27 induces conditions relating to inflammatory diseases of the airways, including steroid-resistant asthma. In some embodiments, biological activity of IL-27 includes any biological activity resulting from IL-27 mediated signaling.

The term “antagonist” is used in the broadest sense, and includes any molecule that partially or fully inhibits or neutralizes a biological activity of a polypeptide, such as IL-27, or that partially or fully inhibits the transcription or translation of a nucleic acid encoding the polypeptide. Exemplary antagonist molecules include, but are not limited to, antagonist antibodies, polypeptide fragments, oligopeptides, organic molecules (including small molecules), and anti-sense nucleic acids.

The term “IL-27 antagonist” refers to a molecule that interacts with at least one factor selected from IL-27 heterodimer, p28, EBI3, IL-27 receptor (IL-27R) heterodimer, WSX-1, and gp130, and inhibits IL-27-mediated signaling. Exemplary IL-27 antagonists include antibodies that bind IL-27 heterodimer, antibodies that bind p28, antibodies that bind EBI3, antibodies that bind IL-27R heterodimer, antibodies that bind WSX-1, WSX-1 extracellular domains (ECDs), and WSX-1 ECD fusion molecules. In some embodiments, an IL-27 antagonist is an antibody that binds to IL-27 heterodimer. In some embodiments, the IL-27 antibody that binds to the IL-27 heterodimer binds to p28 subunit of IL-27, but not to EBI3 subunit of IL-27. In some embodiments, the IL-27 antibody that binds to p28 but not EBI3 blocks binding of IL-27 heterodimer to IL-27R. In some embodiments, an IL-27 antagonist blocks binding of IL-27 to IL-27R.

In some embodiments, an IL-27 antagonist is considered to “inhibit IL-27-mediated signaling” when it reduces expression of CXCL10 in vitro in primary bronchial smooth muscle cells in the presence of TNF-α, IL-27, and fluticasone by at least 50%. See, e.g., Example 1. In some embodiments, an IL-27 antagonist reduced CXCL10 expression in that assay by at least 60%, at least 70%, at least 80%, or at least 90%.

In some embodiments, an IL-27 antagonist is considered to “block binding of IL-27 to IL-27R” when it reduces the amount of detectable binding of IL-27 to IL-27R by at least 50%. In some embodiments, an IL-27 antagonist reduces the amount of detectable binding of IL-27 to IL-27R by at least 60%, at least 70%, at least 80%, or at least 90%. In some such embodiments, the antagonist is said to block ligand binding by at least 50%, at least 60%, at least 70%, etc.

The term “IL-27 antibody” or “antibody that binds IL-27,” as used herein, refers to an antibody (as defined below) that binds to IL-27 heterodimer. In some embodiments, an antibody that binds IL-27 inhibits IL-27-mediated signaling. IL-27 antibodies include antibodies that bind to the IL-27 heterodimer, but not to either p28 or EBI3 alone, antibodies that bind to p28 (alone and/or complexed with EBI3), and antibodies that bind to EBI3 (alone and/or complexed with p28). In some embodiments, an antibody binds to p28, but does not bind to EBI3. In some embodiments, an antibody binds to EBI3, but does not bind to p28. In some embodiments, an IL-27 antibody blocks binding of IL-27 to IL-27R. In some embodiments, anti-IL27 antibody refers to an antibody that is capable of binding IL-27 with sufficient affinity such that the antibody is useful as a diagnostic and/or therapeutic agent in targeting IL-27. In one embodiment, the extent of binding of an anti-IL-27 antibody to an unrelated, non-IL-27 protein is less than about 10% of the binding of the antibody to IL-27 as measured, e.g., by a radioimmunoassay (RIA). In some embodiments, an antibody that binds to IL-27 has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M). In some embodiments, an anti-IL-27 antibody binds to an epitope of IL-27 that is conserved among IL-27 from different species. In some embodiments, an anti-IL-27 antibody binds to the same epitope as a human or humanized anti-IL-27 antibody that binds human IL-27.

The term “p28 antibody” or “antibody that binds p28,” as used herein, refers to an IL-27 antibody that binds to p28. In some embodiments, an antibody that binds p28 inhibits IL-27-mediated signaling. A p28 antibody may bind to p28 alone, to p28 when it is complexed with EBI3, or both. In some embodiments, p28 antibody binds to p28 of IL-27 heterodimer, but does not bind to EBI3. In some embodiments, a p28 antibody prevents association of p28 with EBI3. In some embodiments, a p28 antibody blocks binding of IL-27 to IL-27R, as defined above.

The term “EBI3 antibody” or “antibody that binds EBI3,” as used herein, refers to an IL-27 antibody that binds to EBI3. In some embodiments, an antibody that binds EBI3 inhibits IL-27-mediated signaling. An EBI3 antibody may bind to EBI3 alone, to EBI3 when it is complexed with p28, or both. In some embodiments, an EBI3 antibody prevents association of EBI3 with p28. In some embodiments, an EBI3 antibody blocks binding of IL-27 to IL-27R, as defined above.

The term “IL-27R antibody” or “antibody that binds IL-27R,” as used herein, refers to an antibody that binds to IL-27R heterodimer. In some embodiments, an antibody that binds IL-27R inhibits IL-27-mediated signaling. IL-27R antibodies include antibodies that bind to IL-27R heterodimer, but not to either WSX-1 or gp130 alone, and antibodies that bind to WSX-1 (alone and/or complexed with 130), and antibodies that bind to gp130 (alone and/or complexed with WSX-1). In some embodiments, an IL-27R antibody blocks binding of IL-27 to IL-27R, as defined above.

The term “WSX-1 antibody” or “antibody that binds WSX-1,” as used herein, refers to an IL-27R antibody (as defined below) that binds to WSX-1. In some embodiments, an antibody that binds WSX-1 inhibits IL-27 mediated signaling. A WSX-1 antibody may bind to WSX-1 alone, to WSX-1 when it is complexed with gp130, or both. In some embodiments, a WSX-1 antibody prevents association of WSX-1 and gp130. In some embodiments, a WSX-1 antibody blocks binding of IL-27 to WSX-1, as defined above.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term “antibody” as used herein further refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, and antibodies of various species such as mouse, human, cynomolgus monkey, etc.

In some embodiments, an antibody comprises a heavy chain variable region and a light chain variable region. In some embodiments, an antibody comprises at least one heavy chain comprising a heavy chain variable region and at least a portion of a heavy chain constant region, and at least one light chain comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region” as used herein refers to a region comprising heavy chain CDR1, framework (FR) 2, CDR2, FR3, and CDR3. In some embodiments, a heavy chain variable region also comprises at least a portion of an FR1, which is N-terminal to CDR1, and/or at least a portion of an FR4, which is C-terminal to CDR3.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also includes ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR) 2, CDR2, FR3, and CDR3. In some embodiments, a light chain variable region also comprises an FR1 and/or an FR4.

The term “light chain constant region” as used herein refers to a region comprising a light chain constant domain, C_{L}. Nonlimiting exemplary light chain constant regions include λ, and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

A “CDR-grafted antibody” as used herein refers to a humanized antibody in which the complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The term “WSX-1 extracellular domain” (“WSX-1 ECD”) includes full-length WSX-1 ECDs, WSX-1 ECD fragments, and WSX-1 ECD variants. As used herein, the term “WSX-1 ECD” refers to a WSX-1 polypeptide that lacks the intracellular and transmembrane domains, with or without a signal peptide. The term “full-length WSX-1 ECD”, as used herein, refers to a WSX-1 ECD that extends to the last amino acid of the extracellular domain, and may or may not include an N-terminal signal peptide. In some embodiments, a full-length WSX-1 ECD has the amino acid sequence of SEQ ID NO: 19 (with signal peptide) or SEQ ID NO: 20 (without signal peptide). As used herein, the term “WSX-1 ECD fragment” refers to a WSX-1 ECD having one or more residues deleted from the N and/or C terminus of the full-length ECD and that retains the ability to bind IL-27. The WSX-1 ECD fragment may or may not include an N-terminal signal peptide. As used herein, the term “WSX-1 ECD variants” refers to WSX-1 ECDs that contain amino acid additions, deletions, and substitutions and that remain capable of binding to IL-27. Such variants may be at least 90%, 92%, 95%, 97%, 98%, or 99% identical to the parent WSX-1 ECD. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

The term “WSX-1 ECD fusion molecule” refers to a molecule comprising a WSX-1 ECD, and one or more “fusion partners.” In some embodiment, the WSX-1 ECD and the fusion partner are covalently linked (“fused”). If the fusion partner is also a polypeptide (“the fusion partner polypeptide”), the WSX-1 ECD and the fusion partner polypeptide may be part of a continuous amino acid sequence, and the fusion partner polypeptide may be linked to either the N terminus or the C terminus of the WSX-1 ECD. In such cases, the WSX-1 ECD and the fusion partner polypeptide may be translated as a single polypeptide from a coding sequence that encodes both the WSX-1 ECD and the fusion partner polypeptide (the “WSX-1 ECD fusion protein”). In some embodiments, the WSX-1 ECD and the fusion partner are covalently linked through other means, such as, for example, a chemical linkage other than a peptide bond. Many known methods of covalently linking polypeptides to other molecules (for example, fusion partners) may be used. In other embodiments, the WSX-1 ECD and the fusion partner may be fused through a “linker,” which is comprised of at least one amino acid or chemical moiety.

In some embodiments, the WSX-1 polypeptide and the fusion partner are noncovalently linked. In some such embodiments, they may be linked, for example, using binding pairs. Exemplary binding pairs include, but are not limited to, biotin and avidin or streptavidin, an antibody and its antigen, etc.

Exemplary fusion partners include, but are not limited to, an immunoglobulin Fc domain, albumin, and polyethylene glycol. The amino acid sequences of some exemplary Fc domains are shown in SEQ ID NOs: 11 to 13.

In some embodiments, a WSX-1 ECD amino acid sequence is derived from that of a non-human mammal. In such embodiments, the WSX-1 ECD amino acid sequence may be derived from mammals including, but not limited to, rodents (including mice, rats, hamsters), rabbits, simians, felines, canines, equines, bovines, porcines, ovines, caprines, mammalian laboratory animals, mammalian farm animals, mammalian sport animals, and mammalian pets. WSX-1 ECD fusion molecules incorporating a non-human WSX-1 ECD are termed “non-human WSX-1 ECD fusion molecules.” Similar to the human WSX-1 ECD fusion molecules, non-human fusion molecules may comprise a fusion partner, optional linker, and a WSX-1 ECD. Such non-human fusion molecules may also include a signal peptide. A “non-human WSX-1 ECD fragment” refers to a non-human WSX-1 ECD having one or more residues deleted from the N and/or C terminus of the full-length ECD and that retains the ability to bind to IL-27, p28, and/or EBI3 of the non-human animal from which the sequence was derived. A “non-human WSX-1 ECD variant” refers to WSX-1 ECDs that contain amino acid additions, deletions, and substitutions and that remain capable of binding to IL-27, p28, and/or EBI3 from the animal from which the sequence was derived.

The term “signal peptide” refers to a sequence of amino acid residues located at the N terminus of a polypeptide that facilitates secretion of a polypeptide from a mammalian cell. A signal peptide may be cleaved upon export of the polypeptide from the mammalian cell, forming a mature protein. Signal peptides may be natural or synthetic, and they may be heterologous or homologous to the protein to which they are attached. Exemplary signal peptides include, but are not limited to, the signal peptides of EBI3, p28, WSX-1, and gp130. Exemplary signal peptides also include signal peptides from heterologous proteins. A “signal sequence” refers to a polynucleotide sequence that encodes a signal peptide. In some embodiments, a WSX-1 ECD lacks a signal peptide. In some embodiments, a WSX-1 ECD includes at least one signal peptide, which may be a native WSX-1 signal peptide or a heterologous signal peptide.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “sample” or “patient sample” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate (including, for example, bronchoalveolar lavage fluid and induced sputum); blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of an individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

As used herein, the term “steroid” refers to glucocorticoid-type steroids. Nonlimiting exemplary glucocorticoid-type steroids include prednisone, prednisolone, methylprednisone, fluticasone, budesonide, mometasone, triamcinolone, beclometasone, dexamethasone, and betamethasone.

The term “steroid-resistant [condition]” refers to a subset of a condition that shows an insufficient clinical response to administered steroids, wherein the condition is typically treated with such steroids.

A condition “has previously been characterized as having [a characteristic]” when such characteristic of the condition (e.g., elevated level of at least one protein as described herein) has been shown in at least a subset of patients with the condition, or in one or more animal models of the condition. In some embodiments, such characteristic of the condition does not have to be determined in the patient to be treated with IL-27 antagonist of the present invention. The presence of the characteristic in a specific patient who is to be treated using the present methods and/or compositions need not have been determined in order for the patient to be considered as having a condition that has previously been characterized as having the characteristic.

A “disorder” or “disease” is any condition that would benefit from treatment with an anti-IL27 antagonist of the invention. This includes chronic and acute disorders or diseases including those pathological conditions which predispose the mammal to the disorder in question. Non-limiting examples of disorders to be treated herein include conditions and diseases of the airways, including, but not limited to, airway inflammation, airway hyperresponsiveness, asthma, and COPD.

The term “asthma” refers to an inflammatory disease of the airways that is characterized by recurring and variable symptoms, reversible airflow obstruction, bronchospasm, and airway hyperresponsiveness. Nonlimiting exemplary symptoms of asthma include wheezing, chest tightness, shortness of breath, excess mucus production, and coughing. In some embodiments, asthma is steroid-resistant.

The term “Th2-low asthma” refers to asthma that is characterized by low expression of IL-5 and IL-13 mRNAs, as determined by qPCR. “Low expression” means expression levels of IL-5 and IL-13 that are similar to expression levels of IL-5 and IL-13 in healthy subjects. Expression of IL-5 and IL-13 can be determined by the methods described, e.g., in Woodruff et al. Am. J. Respir. Crit. Care Med. 180: 388-395 (2009).

The term “chronic obstructive pulmonary disease” or “COPD” refers to a progressive disease characterized by difficulty breathing, coughing that produces a large amount of mucus, wheezing, shortness of breath, and/or chest tightness. COPD is typically caused by cigarette smoking and/or long-term exposure to other lung irritants, such as air pollution, chemical fumes, or dust. COPD includes both emphysema and chronic bronchitis. COPD is typically steroid-resistant.

The term “systemic lupus erythematosus” (“lupus” or “SLE”) refers to an autoimmune disorder in which a patient's immune system produces auto-antibodies, causing widespread inflammation and tissue damage. SLE can affect many systems and tissues, including joints, skin, brain, lungs, kidneys, and blood vessels, and patients with SLE may experience fatigue, pain, swelling in their joints, skin rashes, and fevers. In some embodiments, SLE is steroid-resistant.

The term “inflammatory bowel disease” (“IBD”) refers to a group of chronic intestinal diseases characterized by inflammation of the bowel (both the large and small intestine). Nonlimiting exemplary inflammatory bowel diseases include ulcerative colitis, characterized by inflammation of the mucosa (inner lining) of the intestine, and Crohn's disease, characterized by inflammation throughout the bowel wall. While IBD may be limited to the intestine, it can also affect the skin, joints, spine, liver, eyes, and other organs. In some embodiments, IBD is steroid-resistant.

“Treatment,” as used herein, covers any administration or application of a therapeutic for a disease (also referred to herein as a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process.

In some embodiments, asthma or COPD is considered to be treated when patient's forced expiratory volume (or “FEV1”) increases by at least 12%, or increases by at least 200 mL, whichever is less, following administration of an IL-27 antagonist described herein. A normal FEV1 is considered to be 80% or greater of predicted FEV1. Methods of predicting FEV1 are known in the art. Further, a patient's FEV1 can be determined using standard spirometry methods.

The term “effective amount” or “therapeutically effective amount” refers to an amount of a drug effective to treat a disease or disorder in a subject. In certain embodiments, an effective amount refers to an amount effective, at dosages and for periods of time necessary, to achieve the desired therapeutic or prophylactic result. A therapeutically effective amount of an IL-27 antagonist of the invention may vary according to factors such as the disease state, age, sex, and weight of the individual, and the ability of the IL-27 antagonist, to elicit a desired response in the individual. A therapeutically effective amount encompasses an amount in which any toxic or detrimental effects of the IL-27 antagonist are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The terms “inhibition” or “inhibit” refer to a decrease or cessation of any phenotypic characteristic or to the decrease or cessation in the incidence, degree, or likelihood of that characteristic.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

### Therapeutic Compositions and Methods

Methods of Treating Diseases Using IL-27 Antagonists

IL-27 antagonists are provided for use in methods of treating humans and other animals. Methods of treating a disease comprising administering IL-27 antagonists to humans and other animals are provided. In some embodiments, an IL-27 antagonist is used to treat a steroid-resistant disease. Nonlimiting exemplary steroid-resistant diseases that may be treated with IL-27 antagonists, including steroid-resistant asthma, Th2-low asthma, COPD, steroid-resistant systemic lupus erythematosus (SLE), and steroid-resistant inflammatory bowel disease. Nonlimiting exemplary diseases that can be treated with IL-27 antagonists also include steroid-resistant multiple sclerosis and steroid-resistant rheumatoid arthritis. In some embodiments, “treating” a disease comprises alleviating one or more symptoms of the disease, either temporarily or permanently. In some embodiments, permanent alleviation of symptoms occurs with regular dosing of an IL-27 antagonist. Cessation of IL-27 antagonist treatment, in some embodiments, may result in a resumption of one or more symptoms of the disease.

In some embodiments, a method of treating a steroid-resistant disease comprises administering an IL-27 antibody to a subject, wherein the IL-27 antibody inhibits IL-27 mediated signaling. In some embodiments, a method of treating a steroid-resistant disease comprises administering a p28 antibody to a subject, wherein the p28 antibody inhibits IL-27 mediated signaling. In some embodiments, the p28 antibody binds to IL-27 heterodimer. In some embodiments, the p28 antibody binds to p28 subunit of IL-27 heterodimer, but not to EBI3 subunit. In some embodiments, a method of treating a steroid-resistant disease comprises administering an EBI3 antibody to a subject, wherein the EBI3 antibody inhibits IL-27 mediated signaling. In some embodiments, a method of treating a steroid-resistant disease comprises administering an IL-27R antibody to a subject, wherein the IL-27R antibody inhibits IL-27 mediated signaling. In some embodiments, a method of treating a steroid-resistant disease comprises administering a WSX-1 antibody to a subject, wherein the WSX-1 antibody inhibits IL-27 mediated signaling. In some embodiments, a method of treating a steroid-resistant disease comprises administering a WSX-1 ECD fusion molecule to a subject, wherein the WSX-1 ECD fusion molecule inhibits IL-27 mediated signaling. In some embodiments, the disease is selected from steroid-resistant asthma, Th2-low asthma, COPD, steroid-resistant systemic lupus erythematosus (SLE), and steroid-resistant inflammatory bowel disease. In some embodiments, the disease is selected from multiple sclerosis (including steroid-resistant multiple sclerosis) and rheumatoid arthritis (including steroid-resistant rheumatoid arthritis).

In some embodiments, methods of treating steroid-resistant airway inflammation comprising administering an IL-27 antagonist to a subject with steroid-resistant airway inflammation are provided. In some embodiments, methods of treating airway hyperresponsiveness comprising administering an IL-27 antagonist to a subject with airway hyperresponsiveness are provided. In some embodiments, the condition is selected from steroid-resistant asthma, Th2-low asthma, and COPD.

In some embodiments, a method of treating steroid-resistance airway inflammation is provided. In some embodiments, a method of airway hyperresponsiveness, is provided. In some embodiments, airway hyperresponsiveness (also referred to, in some instances, as bronchial hyperresponsivenss) is a condition in which the airways exhibit an exaggerated response to nonspecific stimuli, such as cold air or histamine, resulting in bronchospasms and airway obstruction. See, e.g., Postma et al., Am. J. Respir. Crit. Care Med. 158: S187-S192 (1998). In some embodiments, methods of treating steroid-resistant asthma, Th2-low asthma, and/or COPD are provided. In some embodiments, the method comprises administering an IL-27 antibody to a subject, wherein the IL-27 antibody inhibits IL-27 mediated signaling. In some embodiments, the method comprises administering a p28 antibody to a subject, wherein the p28 antibody inhibits IL-27 mediated signaling. In some embodiments, the method comprises administering an EBI3 antibody to a subject, wherein the EBI3 antibody inhibits IL-27 mediated signaling. In some embodiments, the method comprises administering an IL-27R antibody to a subject, wherein the IL-27R antibody inhibits IL-27 mediated signaling. In some embodiments, the method comprises administering a WSX-1 antibody to a subject, wherein the WSX-1 antibody inhibits IL-27 mediated signaling. In some embodiments, the method comprises administering a WSX-1 ECD fusion molecule to a subject, wherein the WSX-1 ECD fusion molecule inhibits IL-27 mediated signaling.

In some embodiments, a condition to be treated with an IL-27 antagonist has previously been characterized as having an elevated level of at least one, at least two, at least three, at least four, or at least five proteins selected from CXCL9, CXCL10, CXCL11, CD38, and WSX-1 in a subject's bronchial smooth muscle cells. In some embodiments, a condition has previously been characterized as having an elevated level of at least one, at least two, at least three, or at least four protein selected from CXCL9, CXCL10, CD38, and WSX-1 in a subject's bronchial smooth muscle cells. In some embodiments, a condition has previously been characterized as having an elevated level of at least one, at least two, at least three, or at least four protein selected from WSX-1, CXCL9, CXCL10, and CXCL11 in a subject's bronchial epithelial cells. In some embodiments, a condition has previously been characterized as having an elevated level of at least one or at least two proteins selected from CXCL9, and CXCL10 in a subject's bronchial epithelial cells.

CXCL9, CXCL10, and CXCL11 are chemokines that act as T-cell chemoattractants, binding to CXCR3 receptor, which is a receptor found predominantly on Th1 cells.

In some embodiments, a condition has previously been characterized as having an elevated level of at least one protein selected from IL-27 heterodimer, p28, TNF-α, and an interferon (such as IFN-α or IFN-γ) in a sample from a subject's lung. In some embodiments, the sample is selected from a bronchoalveolar lavage sample and a sputum sample (including, but not limited to, an induced sputum sample). In some embodiments, a condition has previously been characterized as having an elevated level of at least one protein selected from IL-27 heterodimer, p28, TNF-α, and an interferon (such as IFN-α or IFN-γ) in at least one cell type from a subject's lung. In some embodiments, at least one cell type is a macrophage.

In some embodiments, steroid-resistant asthma is asthma in which the patient has persistent airway inflammation despite treatment with high dose steroids and/or long term oral steroid treatment. In some embodiments, steroid-resistant asthma is asthma in which the lung function of the patient does not improve following seven days of high-dose (at least 40 mg per day) oral steroid therapy. In some embodiments, steroid-resistant asthma is asthma that requires oral steroids at least 50% of the time over the course of a year and/or requires high-dose inhaled steroids, in order to control the asthma to a level of mild to moderate persistent asthma. In some such embodiments, high dose inhaled steroid treatment is >1,260 mg/dose beclomethasone dipropionate; >1,200 mg/dose budesonide; >2,000 mg/dose fluticasone propionate; or >2,000 mg/dose triamcinolone acetonide.

In some instances, steroid treatment in the absence of an IL-27 antagonist treats acute symptoms of COPD, but does not treat chronic symptoms, such as progressive decline in lung function. In some embodiments, steroid treatment in combination with an IL-27 antagonist treats one or more chronic symptoms of COPD, such as by reducing the progressive decline in lung function, reducing dyspnea, and/or reducing dyspnea on exertion.

In some embodiments, steroid-resistant SLE is SLE that shows no clinical improvement or change in disease activity after treatment with high dose steroids. In some embodiments, high dose steroids in the context of SLE is at least 20 mg per day of oral prednisone for 14 days or longer, or a pharmacologically equivalent dose of another steroid for 14 days or longer.

In some embodiments, steroid-resistant inflammatory bowel disease (IBD) is IBD in which there is little or no clinical improvement in symptoms after treatment with steroids for 2 weeks. In some such embodiments, steroid-resistant IBD shows little or no clinical improvement in symptoms after treatment with high dose steroids for 2 weeks. High dose steroid treatment includes, in some embodiments, treatment with greater than 40 mg prednisone or prednisolone per day. In some embodiments, steroid-resistant IBD shows little or no clinical improvement in symptoms after treatment with intravenous steroids. In some embodiments, intravenous steroids are administered at 0.5-0.75 mg/kg/day prednisone equivalent, such as, for example, 100 mg hydrocortisone every 8 hours or 40 mg methylprednisone per day.

In some embodiments, an IL-27 antagonist is used to treat steroid-resistant rheumatoid arthritis (RA). RA is a chronic autoimmune disease characterized primarily by inflammation of the lining (synovium) of the joints, which can lead to joint damage, resulting in chronic pain, loss of function, and disability. Because RA can affect multiple organs of the body, including skin, lungs, and eyes, it is referred to as a systemic illness.

In some embodiments, an IL-27 antagonist is used to treat steroid-resistant multiple sclerosis (MS). MS is a chronic, autoimmune, demyelinating disease of the CNS in which the body generates antibodies and white blood cells against the cells that produce the myelin sheath. Demyelination occurs when the myelin sheath becomes inflamed, injured, and detaches from the nerve fiber.

Routes of Administration and Carriers

In various embodiments, IL-27 antagonists may be administered subcutaneously, intravenously, or by inhalation. In some embodiments, an IL-27 antagonist may be administered in vivo by various routes, including, but not limited to, oral, intra-arterial, parenteral, intranasal, intramuscular, intracardiac, intraventricular, intratracheal, buccal, rectal, intraperitoneal, intradermal, topical, transdermal, and intrathecal, or otherwise, e.g., by implantation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. In some embodiments, an IL-27 antagonist is delivered using gene therapy. As a non-limiting example, a nucleic acid molecule encoding an IL-27 antagonist may be coated onto gold microparticles and delivered intradermally by a particle bombardment device, or “gene gun,” e.g., as described in the literature (see, e.g., Tang et al., Nature 356:152-154 (1992)).

In various embodiments, compositions comprising IL-27 antagonists are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising IL-27 antagonists may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

Pharmaceutical dosage packs comprising one or more containers, each containing one or more doses of an IL-27 antagonist, are also provided. In some embodiments, a unit dosage is provided wherein the unit dosage contains a predetermined amount of a composition comprising an IL-27 antagonist, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In some embodiments, IL-27 antagonists may be administered in an amount in the range of about 50 μg/kg body weight to about 50 mg/kg body weight per dose. In some embodiments, the IL-27 antagonist is an antibody. In some embodiments, IL-27 antagonists, including antibodies, may be administered in an amount in the range of about 100 μg/kg body weight to about 50 mg/kg body weight per dose. In some embodiments, IL-27 antagonists may be administered in an amount in the range of about 100 μg/kg body weight to about 20 mg/kg body weight per dose. In some embodiments, IL-27 antagonists may be administered in an amount in the range of about 0.5 mg/kg body weight to about 20 mg/kg body weight per dose.

The IL-27 antagonist compositions may be administered as needed to subjects. In some embodiments, an effective dose of an IL-27 antagonist is administered to a subject one or more times. In various embodiments, an effective dose of an IL-27 antagonist is administered to the subject once a month, less than once a month, such as, for example, every two months, every three months, or every six months. In other embodiments, an effective dose of an IL-27 antagonist is administered more than once a month, such as, for example, every three weeks, every two weeks, every week, twice per week, three times per week, daily, or multiple times per day. An effective dose of an IL-27 antagonist is administered to the subject at least once. In some embodiments, the effective dose of an IL-27 antagonist may be administered multiple times, including for periods of at least a month, at least six months, or at least a year. In some embodiments, an IL-27 antagonist is administered to a subject as-needed to alleviate one or more symptoms of a condition.

Combination Therapy

IL-27 antagonists may be administered alone or with other modes of treatment. They may be provided before, substantially contemporaneous with, or after other modes of treatment, for example, smooth muscle ablation therapy, intravenous immunoglobulin, or plasmaphoresis. For treatment of steroid-resistant asthma and/or COPD, IL-27 antagonists may be administered with other therapeutic agents, such as anti-inflammatory drugs and/or bronchodilators. Nonlimiting exemplary anti-inflammatory drugs include steroids, such as prednisone, prednisolone, methylprednisone, fluticasone, budesonide, mometasone, triamcinolone, beclometasone, dexamethasone, and betamethasone; mast cell stabilizers, such as cromoglicic acid, nedocromil sodium; leukotriene antagonists, such as montelukast, zafirlukast, and zileuton; and other anti-inflammatory drugs, such as omalizumab (Xolair®), roflumilast, cilomilast. Nonlimiting exemplary bronchodilators include β₂ agonists, such as albuterol, terbutaline, slameterol, and formoterol; and anticholinergics, such as ipratropium and tiotropium; and other agents such as theophylline.

For treatment of steroid-resistant systemic lupus erythematosus (SLE), IL-27 antagonists may be administered with other therapeutic agents, such as nonsteroidal anti-inflammatory drugs (NSAIDs), including, but not limited to, ibuprofen, naproxen sodium, aspirin, and sulindac; steroids, including, but not limited to, prednisone and methylprednisone; immunosuppressants, including, but not limited to, methotrexate, azathioprine, cyclosporine, chlorambucil, belimumab, and cyclophosphamide; and other drugs, such as mycophenolate mofetil and rituximab (Rituxan®).

For treatment of steroid-resistant inflammatory bowel disease, IL-27 antagonists may be administered with other therapeutic agents, such as steroids, including, but not limited to, prednisone and methylprednisone; immunosuppressants, such as TNF-αinhibitors, antagonists of IL-23, antagonists of IL-17, natalizumab, azathioprine, methotrexate, and 6-mercaptopurine; and mesalamine, an anti-inflammatory.

### IL-27 Antibodies and WSX-1 Antibodies

In some embodiments, antibodies that block binding of IL-27 to WSX-1 are provided. In some embodiments, antibodies that inhibit IL-27 mediated signaling are provided. In some such embodiments, the antibody is an IL-27 antibody. In some embodiments, the IL-27 antibody binds to IL-27 heterodimer. In some embodiments, an IL-27 antibody binds to p28, but does not bind to EBI3. In some embodiments, an IL-27 antibody binds to p28 of the IL-27 heterodimer, but does not bind to EBI3. In some embodiments, an IL-27 antibody binds to EBI3, but not to p28. In some such embodiments, the antibody is a WSX-1 antibody. In some embodiments, an antibody binds to WSX-1 extracellular domain (ECD). In some embodiments, an antibody binds to IL-27 or WSX-1 from multiple species. For example, in some embodiments, an antibody binds to human IL-27 or WSX-1, and also binds to IL-27 or WSX-1 from at least one mammal selected from mouse, rat, dog, guinea pig, and monkey.

Humanized Antibodies

In some embodiments, an IL-27 antibody or a WSX-1 antibody is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

As noted above, a humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also comprises a human heavy chain constant region and/or a human light chain constant region.

Chimeric Antibodies

In some embodiments, an IL-27 or WSX-1 antibody is a chimeric antibody. In some embodiments, an IL-27 or WSX-1 antibody comprises at least one non-human variable region and at least one human constant region. In some such embodiments, all of the variable regions of an IL-27 or WSX-1 antibody are non-human variable regions, and all of the constant regions of the IL-27 or WSX-1 antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

Human Antibodies

In some embodiments, an IL-27 antibody or a WSX-1 antibody is a human antibody. Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

Human Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region, for example, human IgG1, IgG2, IgG3, or IgG4. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1): 105-108 (1993). In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or immune disorders, such as asthma, COPD, SLE, and inflammatory bowel disease. In some such embodiments, a human IgG4 or IgG2 heavy chain constant region is selected or engineered. In some embodiments, an IgG4 constant region comprises an S241P mutation.

### Exemplary Properties of Antibodies

Exemplary Properties of IL-27 Antibodies

In some embodiments, an IL-27 antibody is a p28 antibody that binds to p28, either alone or complexed with EBI3, and inhibits IL-27-mediated signaling. In some embodiments, a p28 antibody binds to p28 when it is complexed with EBI3 (and may or may not also bind to p28 alone). In some such embodiments, a p28 antibody blocks binding of IL-27 to IL-27R. In some embodiments, a p28 antibody is an antibody that inhibits binding of IL-27 heterodimer to IL-27R, wherein the p28 antibody binds to p28 of the IL-27 heterodimer, but does not bind to EBI3. In some embodiments, a p28 antibody binds to p28 alone and does not bind to p28 when it is complexed with EBI3. In some such embodiments, a p28 antibody blocks binding of p28 to EBI3. In some embodiments, a p28 antibody binds to p28 with a binding affinity (K_{D}) of less than 50 nM, less than 20 nM, less than 10 nM, or less than 1 nM.

In some embodiments, an IL-27 antibody is an EBI3 antibody that binds to EBI3, either alone or complexed with p28, and inhibits IL-27-mediated signaling. In some embodiments, an EBI3 antibody binds to EBI3 when it is complexed with p28 (and may or may not also bind to EBI3 alone). In some such embodiments, an EBI3 antibody blocks binding of IL-27 to IL-27R. In some embodiments, an EBI3 antibody binds to EBI3 alone and does not bind to EBI3 when it is complexed with p28. In some such embodiments, an EBI3 antibody blocks binding of EBI3 to p28. In some embodiments, an EBI3 antibody binds to EBI3 with a binding affinity (K_{D}) of less than 50 nM, less than 20 nM, less than 10 nM, or less than 1 nM.

In some embodiments, an IL-27 antibody binds to the IL-27 heterodimer, but does not bind to p28 alone or to EBI3 alone, and inhibits IL-27-mediated signaling. In some embodiments, an IL-27 antibody binds to IL-27 with a binding affinity (K_{D}) of less than 50 nM, less than 20 nM, less than 10 nM, or less than 1 nM. In some embodiments, an IL-27 antibody blocks binding of IL-27 to IL-27R.

Exemplary Properties of IL-27R Antibodies

In some embodiments, an IL-27R antibody binds to the IL-27R heterodimer, but does not bind to WSX-1 alone or to gp130 alone, and inhibits IL-27-mediated signaling. In some embodiments, an IL-27R antibody binds to IL-27R with a binding affinity (K_{D}) of less than 50 nM, less than 20 nM, less than 10 nM, or less than 1 nM. In some embodiments, an IL-27R antibody blocks binding of IL-27 to IL-27R.

In some embodiments, an IL-27 antibody is a WSX-1 antibody that binds to WSX-1, either alone or complexed with gp130, and inhibits IL-27-mediated signaling. In some embodiments, a WSX-1 antibody binds to WSX-1 when it is complexed with gp130 (and may or may not also bind to WSX-1 alone). In some such embodiments, a WSX-1 antibody blocks binding of IL-27 to IL-27R. In some embodiments, a WSX-1 antibody binds to WSX-1 alone and does not bind to WSX-1 when it is complexed with gp130. In some such embodiments, a WSX-1 antibody blocks binding of WSX-1 to gp130. In some embodiments, a WSX-1 antibody binds to WSX-1 with a binding affinity (K_{D}) of less than 50 nM, less than 20 nM, less than 10 nM, or less than 1 nM.

### Antibody Conjugates

In some embodiments, an IL-27 or WSX-1 antibody is conjugated to a label. As used herein, a label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

In some embodiments, a label is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label is a polypeptide, the label can be expressed from the same expression vector with at least one antibody chain to produce a polypeptide comprising the label fused to an antibody chain.

### WSX-1 Extracellular Domains (ECDs)

Nonlimiting exemplary WSX-1 ECDs include full-length WSX-1 ECDs, WSX-1 ECD fragments, and WSX-1 ECD variants. WSX-1 ECDs bind to IL-27. In some embodiments, a WSX-1 ECD inhibits IL-27 mediated signaling. In some embodiments, a WSX-1 ECD does not associate with gp130. WSX-1 ECDs may include or lack a signal peptide. Exemplary WSX-1 ECDs include, but are not limited to, human WSX-1 ECDs having amino acid sequences selected from SEQ ID NOs.: 9 and 19 (with signal peptide) and 10 and 20 (without signal peptide). In some embodiments, a human WSX-1 ECD ends at amino acid 512, 514, 516, or 522, counting from the first amino acid of the signal peptide. Nonlimiting exemplary WSX-1 ECDs are described, e.g., in U.S. Publication Nos. US 2008/0038223, US 2010/0092465, and US 2009/0280082, and references cited therein.

WSX-1 ECD fragments include fragments comprising deletions at the N- and/or C-terminus of the full-length WSX-1 ECD, wherein the WSX-1 ECD fragment retains the ability to bind IL-27. WSX-1 ECD fragments may include or lack a signal peptide. Exemplary WSX-1 ECD fragments include, but are not limited to, the amino acid sequence of SEQ ID NO.: 10 (without signal peptide) or SEQ ID NO.: 9 (with signal peptide).

WSX-1 ECD variants include variants comprising one or more amino acid additions, deletions, and/or substitutions, and that remain capable of binding IL-27. In some embodiments, a WSX-1 ECD variant sequence is at least 90%, 92%, 95%, 97%, 98%, or 99% identical to the corresponding sequence of the parent WSX-1 ECD.

### Fusion Partners and Conjugates

In some embodiments, a WSX-1 ECD of the present invention may be combined with a fusion partner polypeptide, resulting in a WSX-1 ECD fusion protein. These fusion partner polypeptides may facilitate purification, and the WSX-1 ECD fusion proteins may show an increased half-life in vivo. Fusion partner polypeptides that have a disulfide-linked dimeric structure due to the IgG portion may also be more efficient in binding and neutralizing other molecules than the monomeric WSX-1 ECD fusion protein or the WSX-1 ECD alone. Suitable fusion partners of a WSX-1 ECD include, for example, polymers, such as water soluble polymers, the constant domain of immunoglobulins; all or part of human serum albumin (HSA); fetuin A; fetuin B; a leucine zipper domain; a tetranectin trimerization domain; mannose binding protein (also known as mannose binding lectin), for example, mannose binding protein 1; and an Fc region, as described herein and further described in U.S. Pat. No. 6,686,179.

A WSX-1 ECD fusion molecule may be prepared by attaching polyaminoacids or branch point amino acids to the WSX-1 ECD. For example, the polyaminoacid may be a carrier protein that serves to increase the circulation half life of the WSX-1 ECD (in addition to the advantages achieved via a fusion molecule). For the therapeutic purpose of the present invention, such polyaminoacids should ideally be those that do not create neutralizing antigenic response, or other adverse responses. Such polyaminoacids may be chosen from serum album (such as HSA), an additional antibody or portion thereof, for example the Fc region, fetuin A, fetuin B, leucine zipper nuclear factor erythroid derivative-2 (NFE2), neuroretinal leucine zipper, tetranectin, or other polyaminoacids, for example, lysines. As described herein, the location of attachment of the polyaminoacid may be at the N terminus or C terminus, or other places in between, and also may be connected by a chemical linker moiety to the selected molecule.

Polymers

Polymers, for example, water soluble polymers, may be useful in the present invention to reduce precipitation of the WSX-1 ECD to which the polymer is attached in an aqueous environment, such as typically found in a physiological environment. Polymers employed in the invention will be pharmaceutically acceptable for the preparation of a therapeutic product or composition.

Suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly (β-amino acids) (either homopolymers or random copolymers), poly(n-vinyl pyrrolidone) polyethylene glycol, polypropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll, or dextran and mixtures thereof.

Polymers used herein, for example water soluble polymers, may be of any molecular weight and may be branched or unbranched. In some embodiments, the polymers have an average molecular weight of between 2 kDa and 100 kDa, between 5 kDa and 50 kDa, or between 12 kDa and 25 kDa. Generally, the higher the molecular weight or the more branches, the higher the polymer:protein ratio. Other sizes may also be used, depending on the desired therapeutic profile; for example, the duration of sustained release; the effects, if any, on biological activity; the ease in handling; the degree or lack of antigenicity; and other known effects of a polymer on a WSX-1 ECD of the invention.

In some embodiments, the present invention contemplates the chemically derivatized WSX-1 ECD to include mono- or poly- (e.g., 2-4) PEG moieties. Pegylation may be carried out by any of the pegylation reactions available. There are a number of PEG attachment methods available to those skilled in the art. See, for example, EP 0 401 384; Malik et al., Exp. Hematol., 20:1028-1035 (1992); Francis, Focus on Growth Factors, 3(2):4-10 (1992); EP 0 154 316; EP 0 401 384; WO 92/16221; WO 95/34326; Chamow, Bioconjugate Chem., 5:133-140 (1994); U.S. Pat. No. 5,252,714; and the other publications cited herein that relate to pegylation.

Markers

WSX-1 ECDs of the present invention may be fused to marker sequences, such as a peptide that facilitates purification of the fused polypeptide. The marker amino acid sequence may be a hexa-histidine peptide such as the tag provided in a pQE vector (Qiagen, Mississauga, Ontario, Canada), among others, many of which are commercially available. As described in Gentz et al., Proc. Natl. Acad. Sci. 86:821-824 (1989), for instance, hexa-histidine provides for convenient purification of the fusion protein. Another peptide tag useful for purification, the hemagglutinin (HA) tag, corresponds to an epitope derived from the influenza HA protein. (Wilson et al., Cell 37:767 (1984)). Any of these above fusions may be engineered using the WSX-1 ECDs of the present invention.

Oligomerization Domain Fusion Partners

In various embodiments, oligomerization offers some functional advantages to a fusion protein, including, but not limited to, multivalency, increased binding strength, and the combined function of different domains. Accordingly, in some embodiments, a fusion partner comprises an oligomerization domain, for example, a dimerization domain. Exemplary oligomerization domains include, but are not limited to, coiled-coil domains, including alpha-helical coiled-coil domains; collagen domains; collagen-like domains; and certain immunoglobulin domains. Exemplary coiled-coil polypeptide fusion partners include, but are not limited to, the tetranectin coiled-coil domain; the coiled-coil domain of cartilage oligomeric matrix protein; angiopoietin coiled-coil domains; and leucine zipper domains. Exemplary collagen or collagen-like oligomerization domains include, but are not limited to, those found in collagens, mannose binding lectin, lung surfactant proteins A and D, adiponectin, ficolin, conglutinin, macrophage scavenger receptor, and emilin.

Antibody Fc Immunoglobulin Domain Fusion Partners

Many Fc domains that may be used as fusion partners are known in the art. In some embodiments, a fusion partner is an Fc immunoglobulin domain. An Fc fusion partner may be a wild-type Fc found in a naturally occurring antibody, a variant thereof, or a fragment thereof. Non-limiting exemplary Fc fusion partners include Fcs comprising a hinge and the CH2 and CH3 constant domains of a human IgG, for example, human IgG1, IgG2, IgG3, or IgG4. In some embodiments, an Fc fusion partner comprises a C237S mutation, for example, in an IgG1 constant region. See, e.g., SEQ ID NO: 11. In some embodiments, an Fc fusion partner is a human IgG4 constant region. In some such embodiments, the human IgG4 constant region comprises an S241P mutation. See, e.g., Angal et al. Mol. Immunol. 30(1): 105-108 (1993). In some embodiments, an Fc fusion partner comprises a hinge, CH2, and CH3 domains of human IgG2 with a P331S mutation, as described in U.S. Pat. No. 6,900,292. Additional exemplary Fc fusion partners also include, but are not limited to, human IgA and IgM. Certain exemplary Fc domain fusion partners are shown in SEQ ID NOs: 11 to 13.

In some embodiments, effector function is not desirable. For example, in some embodiments, effector function may not be desirable in treatments of inflammatory conditions and/or immune disorders, such as asthma, COPD, SLE, and inflammatory bowel disease. In some such embodiments, a human IgG4 or IgG2 heavy chain constant region is selected or engineered. In some embodiments, an IgG4 constant region comprises an S241P mutation.

Albumin Fusion Partners and Albumin-Binding Molecule Fusion Partners

In some embodiments, a fusion partner is an albumin. Exemplary albumins include, but are not limited to, human serum album (HSA) and fragments of HSA that are capable of increasing the serum half-life or bioavailability of the polypeptide to which they are fused. In some embodiments, a fusion partner is an albumin-binding molecule, such as, for example, a peptide that binds albumin or a molecule that conjugates with a lipid or other molecule that binds albumin. In some embodiments, a fusion molecule comprising HSA is prepared as described, e.g., in U.S. Pat. No. 6,686,179.

Exemplary Attachment of Fusion Partners

The fusion partner may be attached, either covalently or non-covalently, to the N terminus or the C terminus of the WSX-1 ECD. The attachment may also occur at a location within the WSX-1 ECD other than the N terminus or the C terminus, for example, through an amino acid side chain (such as, for example, the side chain of cysteine, lysine, serine, or threonine).

In either covalent or non-covalent attachment embodiments, a linker may be included between the fusion partner and the WSX-1 ECD. Such linkers may be comprised of at least one amino acid or chemical moiety. Exemplary methods of covalently attaching a fusion partner to a WSX-1 ECD include, but are not limited to, translation of the fusion partner and the WSX-1 ECD as a single amino acid sequence and chemical attachment of the fusion partner to the WSX-1 ECD. When the fusion partner and a WSX-1 ECD are translated as single amino acid sequence, additional amino acids may be included between the fusion partner and the WSX-1 ECD as a linker. In some embodiments, the linker is selected based on the polynucleotide sequence that encodes it, to facilitate cloning the fusion partner and/or WSX-1 ECD into a single expression construct (for example, a polynucleotide containing a particular restriction site may be placed between the polynucleotide encoding the fusion partner and the polynucleotide encoding the WSX-1 ECD, wherein the polynucleotide containing the restriction site encodes a short amino acid linker sequence). When the fusion partner and the WSX-1 ECD are covalently coupled by chemical means, linkers of various sizes may typically be included during the coupling reaction.

Exemplary methods of non-covalently attaching a fusion partner to a WSX-1 ECD include, but are not limited to, attachment through a binding pair. Exemplary binding pairs include, but are not limited to, biotin and avidin or streptavidin, an antibody and its antigen, etc.

### Exemplary Properties of WSX-1 ECDs and WSX-1 ECD Fusion Molecules

In some embodiments, a WSX-1 ECD or a WSX-1 ECD fusion molecule binds to IL-27, and inhibits IL-27-mediated signaling. In some embodiments, a WSX-1 ECD or a WSX-1 ECD fusion molecule binds to IL-27 with a binding affinity (K_{D}) of less than 50 nM, less than 20 nM, less than 10 nM, or less than 1 nM. In some embodiments, a WSX-1 ECD or a WSX-1 ECD fusion molecule blocks binding of IL-27 to IL-27R.

### Additional IL-27 Antagonists

In some embodiments, additional molecules that bind IL-27, p28, EBI3, IL-27R, or WSX-1 are provided. Such molecules include, but are not limited to, non-canonical scaffolds, such as anti-calins, adnectins, ankyrin repeats, etc. See, e.g., Hosse et al., Prot. Sci. 15:14 (2006); Fiedler, M. and Skerra, A., “Non-Antibody Scaffolds,” pp. 467-499 in Handbook of Therapeutic Antibodies, Dubel, S., ed., Wiley-VCH, Weinheim, Germany, 2007.

### Signal Peptides

In order for some secreted proteins to express and secrete in large quantities, a signal peptide from a heterologous protein may be desirable. Employing heterologous signal peptides may be advantageous in that a resulting mature polypeptide may remain unaltered as the signal peptide is removed in the ER during the secretion process. The addition of a heterologous signal peptide may be required to express and secrete some proteins.

Nonlimiting exemplary signal peptide sequences are described, e.g., in the online Signal Peptide Database maintained by the Department of Biochemistry, National University of Singapore. See Choo et al., BMC Bioinformatics, 6: 249 (2005); and PCT Publication No. WO 2006/081430.

### Co-Translational and Post-Translational Modifications

In some embodiments, a polypeptide such as an IL-27 antibody, a WSX-1 antibody, a WSX-1 ECD, or a WSX-1 ECD fusion molecule, is differentially modified during or after translation, for example by glycosylation, sialylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH₄, acetylation; formylation; oxidation; reduction; and/or metabolic synthesis in the presence of tunicamycin.

Additional post-translational modifications encompassed by the invention include, for example, N-linked or O-linked carbohydrate chains; processing of N-terminal or C-terminal ends; attachment of chemical moieties to the amino acid backbone; chemical modifications of N-linked or O-linked carbohydrate chains; and addition or deletion of an N-terminal methionine residue as a result of prokaryotic host cell expression.

### Nucleic Acid Molecules Encoding IL-27 Antagonists

In some embodiments, nucleic acid molecules comprising polynucleotides that encode WSX-1 ECDs or WSX-1 ECD fusion molecules are provided. Nucleic acid molecules comprising polynucleotides that encode WSX-1 ECD fusion molecules in which the WSX-1 ECD and the fusion partner are translated as a single polypeptide are also provided.

In some embodiments, a polynucleotide encoding a WSX-1 ECD comprises a nucleotide sequence that encodes a signal peptide, which, when translated, will be fused to the N-terminus of the WSX-1 ECD. As discussed above, the signal peptide may be the native WSX-1 signal peptide, or may be another heterologous signal peptide. In some embodiments, the nucleic acid molecule comprising the polynucleotide encoding the gene of interest is an expression vector that is suitable for expression in a selected host cell.

Nucleic acid molecules may be constructed using recombinant DNA techniques conventional in the art. In some embodiments, a nucleic acid molecule is an expression vector that is suitable for expression in a selected host cell.

### IL-27 Antagonist Expression and Production

Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

Vectors comprising polynucleotides that encode WSX-1 ECDs are provided. Vectors comprising polynucleotides that encode WSX-1 ECD fusion molecules are also provided. Such vectors include, but are not limited to, DNA vectors, phage vectors, viral vectors, retroviral vectors, etc.

In some embodiments, a vector is selected that is optimized for expression of polypeptides in CHO or CHO-derived cells, or in NSO cells. Exemplary such vectors are described, e.g., in Running Deer et al., Biotechnol. Frog. 20:880-889 (2004).

In some embodiments, a vector is chosen for in vivo expression of an IL-27 antagonist in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Similarly, in various embodiments, WSX-1 ECDs and/or WSX-1 ECD fusion molecules may be expressed in prokaryotic cells, such as bacterial cells; or in eukaryotic cells, such as fungal cells, plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains, light chains, ECDs, and/or ECD fusion molecules. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Purification of IL-27 Antagonists

The antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

WSX-1 ECDs and WSX-1 ECD fusion molecules may be purified by any suitable method. Such methods include, but are not limited to, the use of affinity matrices or hydrophobic interaction chromatography. Suitable affinity ligands include any ligands that bind to WSX-1 (such as IL-27), or that bind to the fusion partner, or antibodies thereto. Further, a Protein A, Protein G, Protein A/G, or an antibody affinity column may be used to bind to an Fc fusion partner to purify a WSX-1 ECD fusion molecule.

In some embodiments, hydrophobic interactive chromatography, for example, a butyl or phenyl column, is also used for purifying some polypeptides. Many methods of purifying polypeptides are known in the art.

Cell-Free Production of IL-27 Antagonists

In some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### In Vitro Screen to Identify Factors Affecting Steroid Sensitivity

In order to identify factors that induce a steroid-resistant state, an assay was set up as follows. Primary human bronchial smooth muscle cells were cultured in the presence of 5 ng/ml TNF-α and 25 nM fluticasone and a test substance. Steroid sensitivity was determined by detecting expression levels of CXCL10 (IP-10) and CD38 using a bDNA assay (QuantiGene® Plex 2.0; Panomics, Santa Clara, Calif.). Expression of each of those genes is suppressed by steroids, such as fluticasone. If a factor induces a steroid-insensitive state, expression of those genes should increase. Interferon family proteins were included as test substances as positive controls.

Using this assay, a library of over 4000 secreted and extracellular domain protein test substances were screened to identify test substances that induce a steroid-resistant state. The test substances were individually expressed in 293T cells and the cell supernatants used to test each substance in the assay.

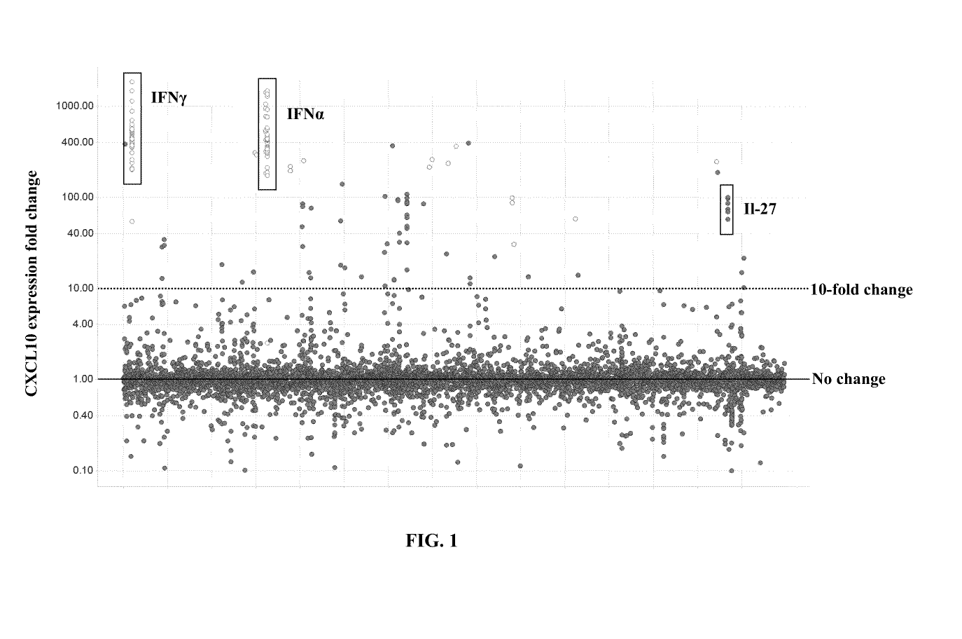

FIG. 1 shows exemplary results from the screen. The fold-change in CXCL10 expression is shown for the test substances. Several clusters of positive data points are evident. One of those clusters resulted from IL-27 as the test substance, and is indicated by a box. Clusters of data points resulting from IFN-γ and IFN-α, positive controls in the screen, are also indicated.

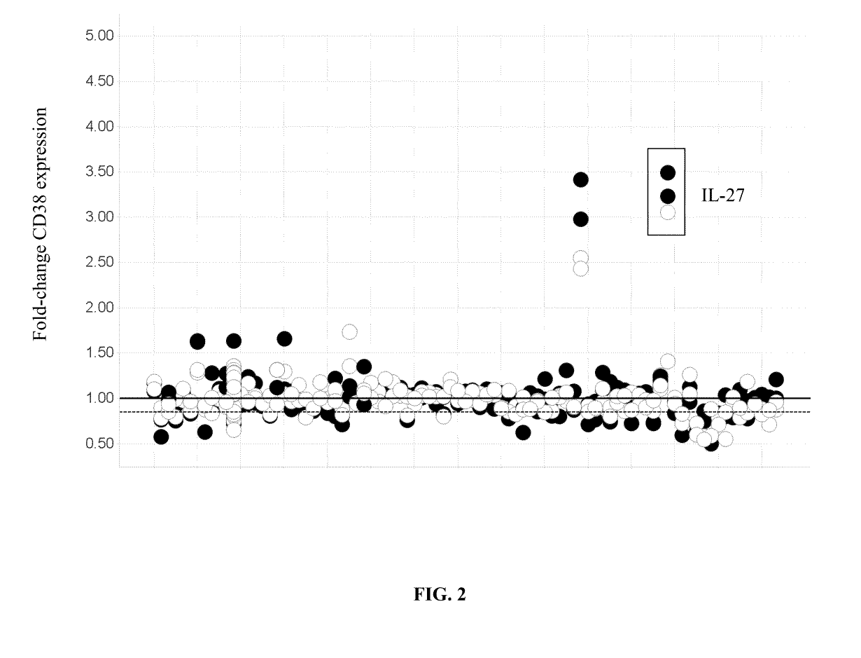

FIG. 2 shows exemplary results from two separate retests of some of the test substances for their effect on CD38 expression. Two clusters of positive data points are evident. One of those clusters, indicated by a box, resulted from IL-27 as the test substance. A similar retest of some of the test substances for their effect on CXCL10 expression also showed a positive cluster of data points resulting from IL-27 (data not shown).

### Example 2

### Dose Dependence of IL-27-Induced Steroid Insensitivity

Primary human bronchial smooth muscle cells were cultured in the presence of 5 ng/ml TNF-α, 25 nM fluticasone, and increasing concentrations of linked IL-27 (Cat. No. 2526-IL; R&D Systems, Minneapolis, Minn.). CXCL10 and CD38 expression levels were determined by bDNA assay.

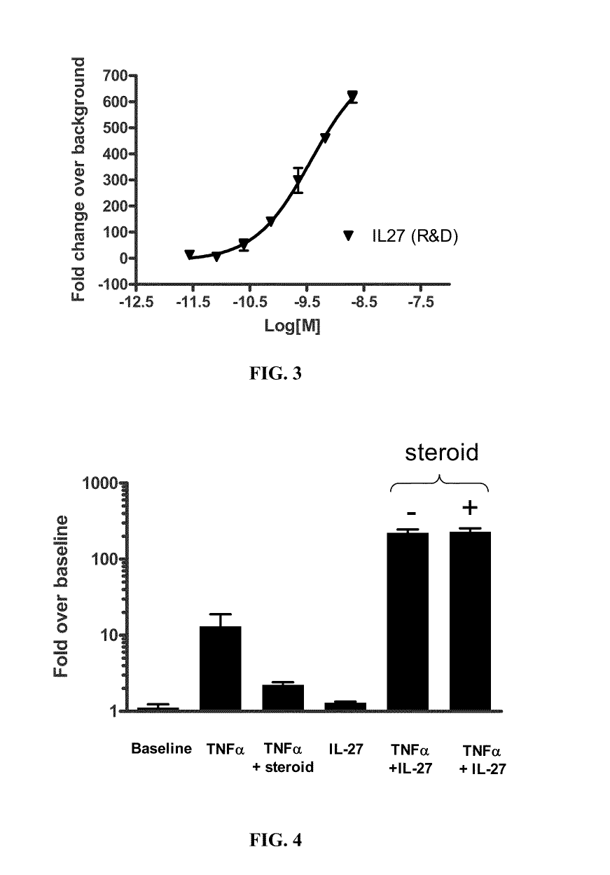

As shown in FIG. 3, IL-27 induced steroid-insensitivity in a dose-dependent manner. Increasing concentrations of IL-27 resulted in increasing expression of CXCL10 in the presence of fluticasone. The EC50 for IL-27 in that experiment was 3.72×10⁻¹⁰ M. Expression of CD38 also increased with increasing concentration of linked IL-27. (Data not shown.)

### Example 3

### Synergistic Upregulation of CXCL10 Expression by TNF-α and IL-27

Primary human bronchial smooth muscle cells were cultured in the presence of 5 ng/ml TNF-αalone, 5 ng/ml TNF-α and 25 nM fluticasone; 1 μg/ml linked IL-27; 5 ng/ml TNF-α and 30 ng/ml IL-27; or 5 ng/ml TNF-α, 25 nM fluticasone, and 30 ng/ml IL-27. CXCL10 expression was determined by bDNA assay.

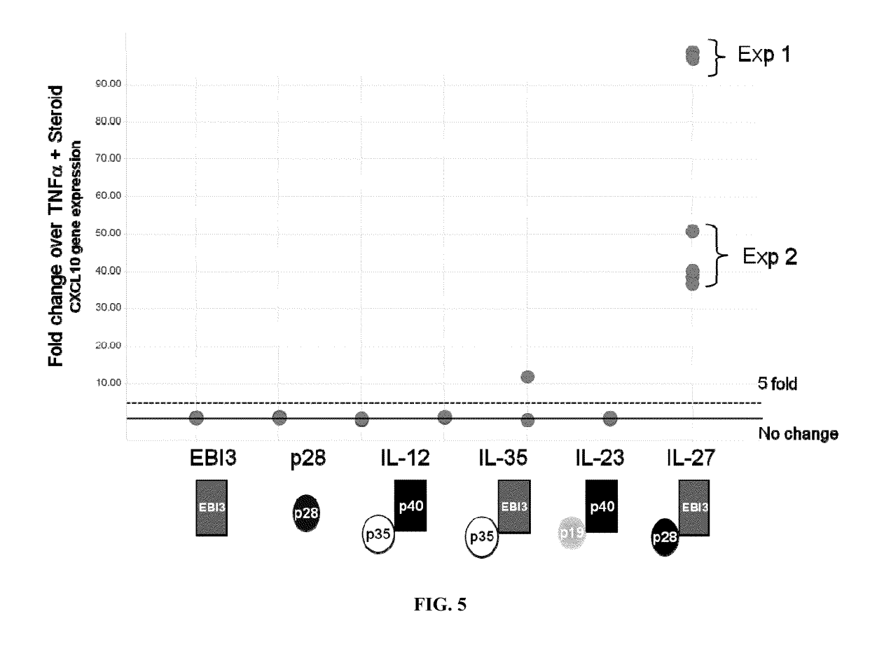

As shown in FIG. 4, TNF-αalone increased expression of CXCL10, which was suppressed by fluticasone. IL-27 alone did not affect expression of CXCL10, but IL-27 in combination with TNF-αresulted in very high CXCL10 expression. Fluticasone had no effect on the high expression of CXCL10 induced by the combination of IL-27 and TNF-α. Finally, IFN-γ alone did not induce CXCL10 expression, and IFN-γ and IL-27 together did not show any synergistic increase in CXCL10 expression in bronchial smooth muscle cells, in contrast to TNF-α and IL-27. (Data not shown.)

### Example 4

### IL-27, But Not Other IL-12 Family Cytokines, Synergizes with TNF-α

Primary human bronchial smooth muscle cells were cultured in the presence of 5 ng/ml TNF-α, 25 nM fluticasone, and a supernatant from 293T cells expressing IL-27 subunit EBI3 alone, IL-27 subunit p28 alone, IL-12 (comprised of p35 and p40), IL-35 (comprised of p35 and EBI3), IL-23 (comprised of p19 and p40), or IL-27 (comprised of p28 and EBI3). CXCL10 expression was determined by bDNA assay.

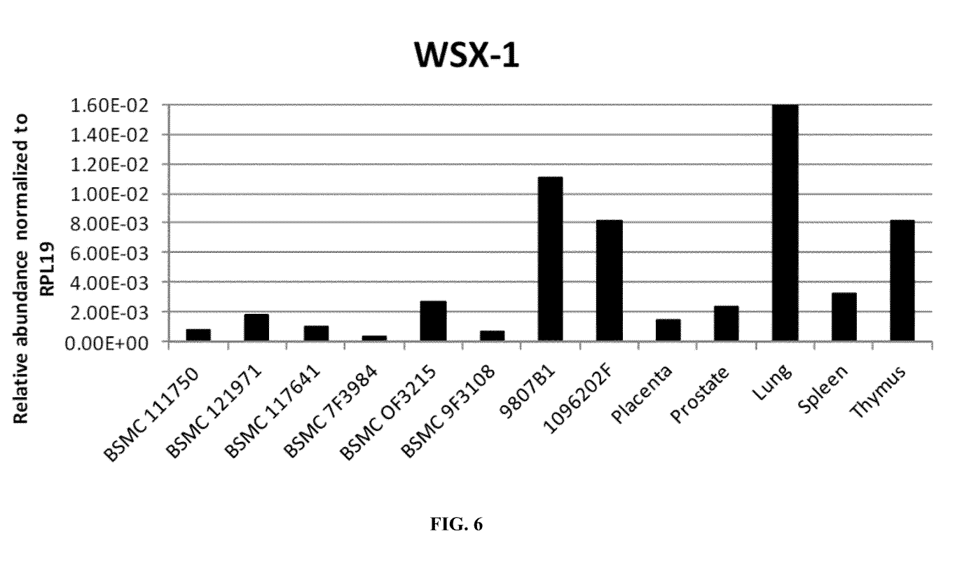

As shown in FIG. 5, only IL-27 synergistically increased CXCL10 expression in the presence of TNF-α and fluticasone.

### Example 5

### IL-27RA (WSX-1) is Upregulated in Lung Cells Contacted with TNF-α

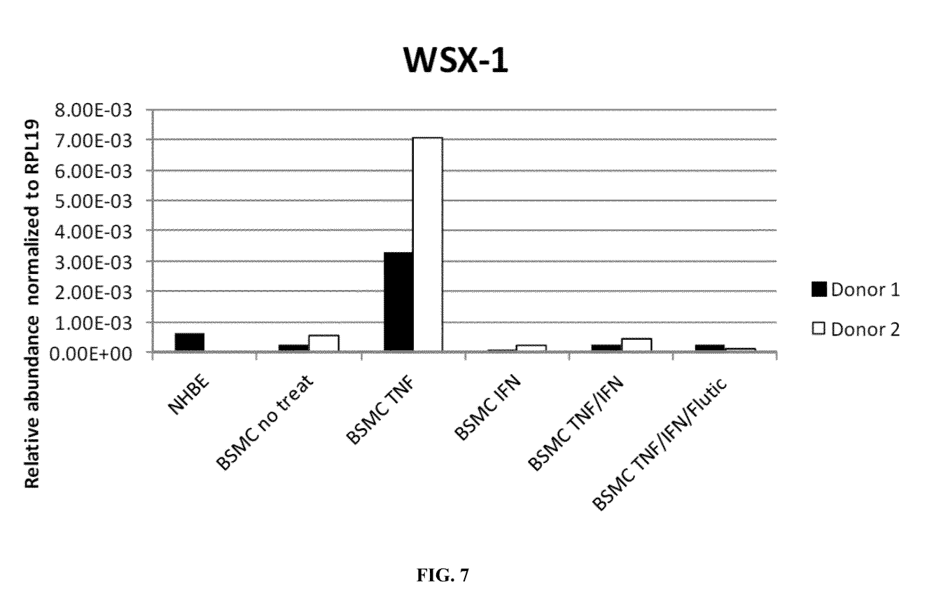

Expression of IL-27 receptor α subunit (also known as WSX-1) was determined in various human tissues and cells, including various primary bronchial smooth muscle cell (BSMC) samples, by quantitative RT-PCR. Normal BSMCs were obtained from Lonza (Walkersville, Md.), tissue RNA was obtained from Clontech (Mountain View, Calif.), and patient samples were obtained from Asterand (Detroit, Mich.). WSX-1 expression levels were normalized to ribosomal protein L19 (RPL19). As shown in FIG. 6, WSX-1 is most highly expressed in lung tissue, including lung tissue from a patient with asthma (sample 1096202F) and a patient with chronic obstructive pulmonary disease (COPD; sample 9807B1). WSX-1 expression was low in various primary bronchial smooth muscle cell samples.

The effect of various factors on WSX-1 expression was then determined in primary bronchial smooth muscle cells (BSMCs). The BSMCs were treated with 5 ng/ml TNF-α, 10 ng/ml IFN-γ, 5 ng/ml TNF-α and 10 ng/ml IFN-γ, or 5 ng/ml TNF-α and 10 ng/ml IFN-γ and 25 nM fluticasone. WSX-1 expression was determined by quantitative RT-PCR and normalized to RPL19.

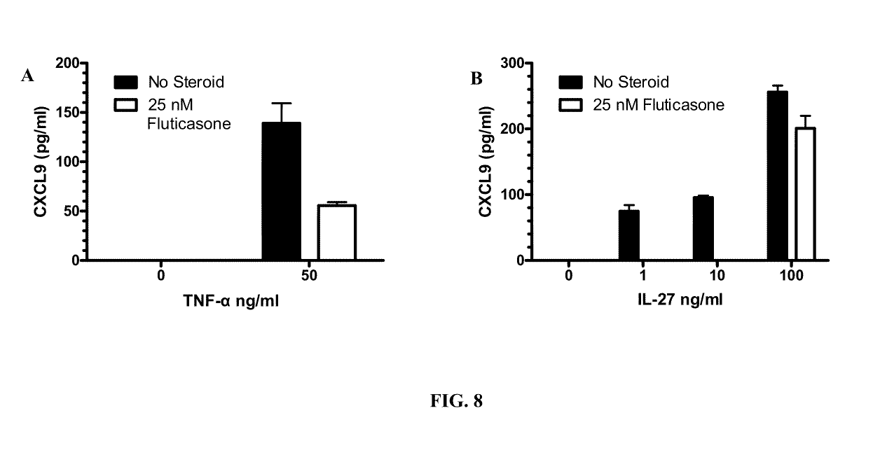

As shown in FIG. 7, WSX-1 expression is up-regulated in BSMCs upon treatment with TNF-α. The figure shows normal human bronchial epithelial cells (NHBE) on the far left, and primary BSMCs from two different patients.

### Example 6

### Induction of CXCL10 and CXCL9 by IL-27 in Human Bronchial Epithelial Cells is Steroid Insensitive

Primary human bronchial epithelial cells from normal and diseased donors (COPD) were cultured under air liquid interface conditions to produce a 3D pseudostratified model of the epithelial cell layer in the lungs. See, e.g., Clonetics™ B-ALI™ Air-Liquid Interface Medium (Lonza, Walkersville, Md.). The cells are cultured in the presence of 50 ng/ml TNF-α and 1, 10, or 100 ng/ml human IL-27. In addition, cells were cultured in the presence of 2 or 10 ng/ml TNF-α, 10 ng/ml human IL-27, with or without 25 nM fluticasone. CXCL9 (MIG) expression was determined by ELISA assay.

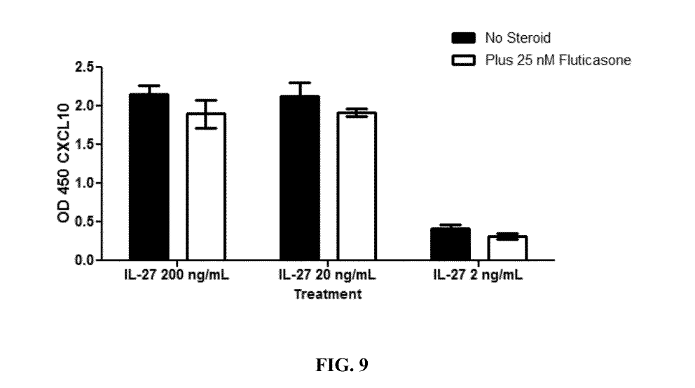

The results of that experiment are shown in FIGS. 8, 9, and 10. FIG. 8A shows induction of the chemokine CXCL9 (MIG) by TNF-α in the presence and absence of 25 nM fluticasone in primary human bronchial epithelial cells from a normal donor. Addition of fluticasone suppressed the TNF-α-mediated induction of MIG expression. FIG. 8B shows induction of MIG by various concentrations of IL-27 in the presence and absence of 25 nM fluticasone in primary human bronchial epithelial cells. At 100 ng/ml IL-27, induction of MIG was relatively insensitive to fluticasone suppression. Fluticasone was only added at the 100 ng/ml concentration of IL-27. Finally, IL-27 induces some expression of CXCL9 and CXCL10 in bronchial epithelial cells, but no synergy was observed with IL-27, in contrast to the combination of IL-27 and TNF-α. (Data not shown.)

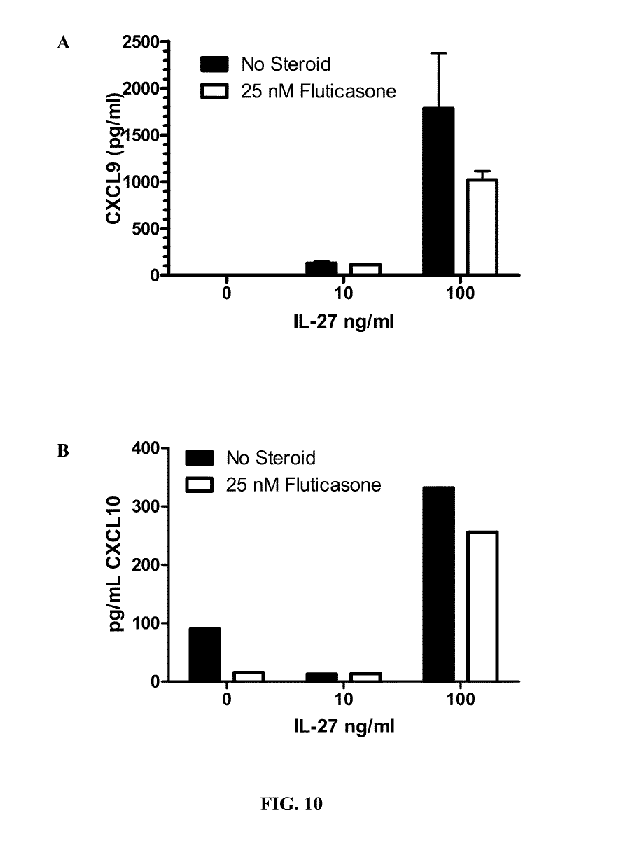

FIG. 9 shows that induction of CXCL10 expression in primary human bronchial epithelial cells from by IL-27 was steroid-insensitive at all concentrations of IL-27.

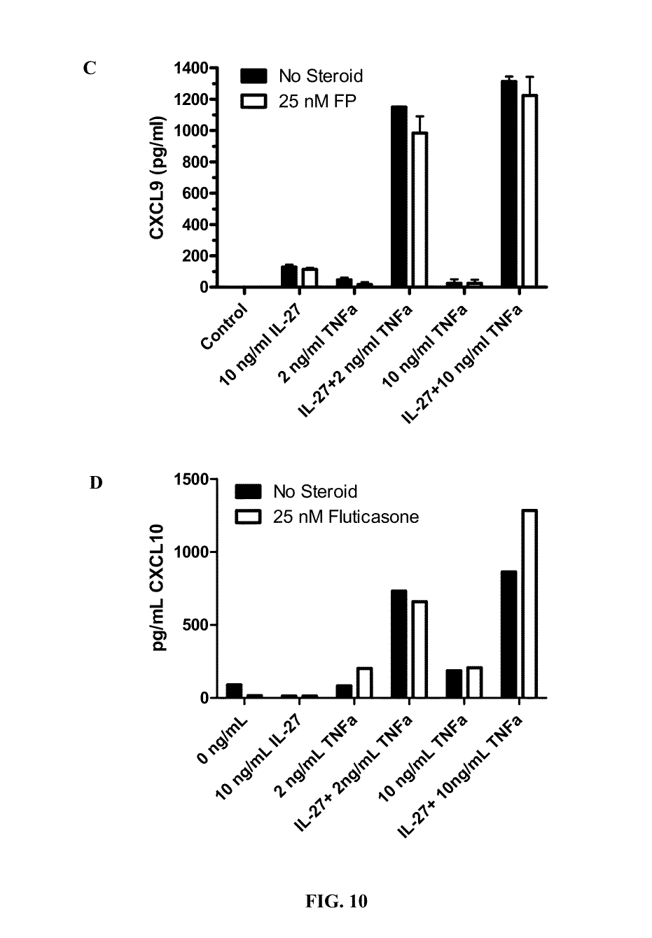

FIG. 10 shows (A) CXCL9 and (B) CXCL10 expression induced by IL-27 in primary human bronchial epithelial cells from a COPD donor at 100 ng/ml. Expression of both chemokines was relatively insensitive to 25 nM fluticasone treatment. As shown in FIGS. 10C and 10D, synergy with TNF-α for both chemokines is also steroid insensitive in primary human bronchial epithelial cells from a COPD donor.

### Example 7

### IL-27 Antagonists Inhibit IL-27-Induced Expression of CXCL10 in Human Bronchial Smooth Muscle Cells

Various concentrations of an IL-27 antagonist, WSX-1 ECD (0.2 to 20 μg/mL), were preincubated with 5 ng/mL of human IL-27 in the presence of 5 ng/mL of human TNF-α. Both linked and native human IL-27 were tested. Human linked IL-27 was obtained from R&D Systems and is produced with a linker between the p28 subunit and the EBI3 subunit. Native IL-27 was produced and purified in house by transfecting mammalian cells with separate vectors expressing each subunit and purifying the resulting p28/EBI3 (IL-27) heterodimer from the cell culture supernatant. Following the preincubation of WSX-1 ECD with IL-27 and TNF-α, the stimulus was added to primary human bronchial smooth muscle cells in culture. CXCL10 expression was determined by ELISA.

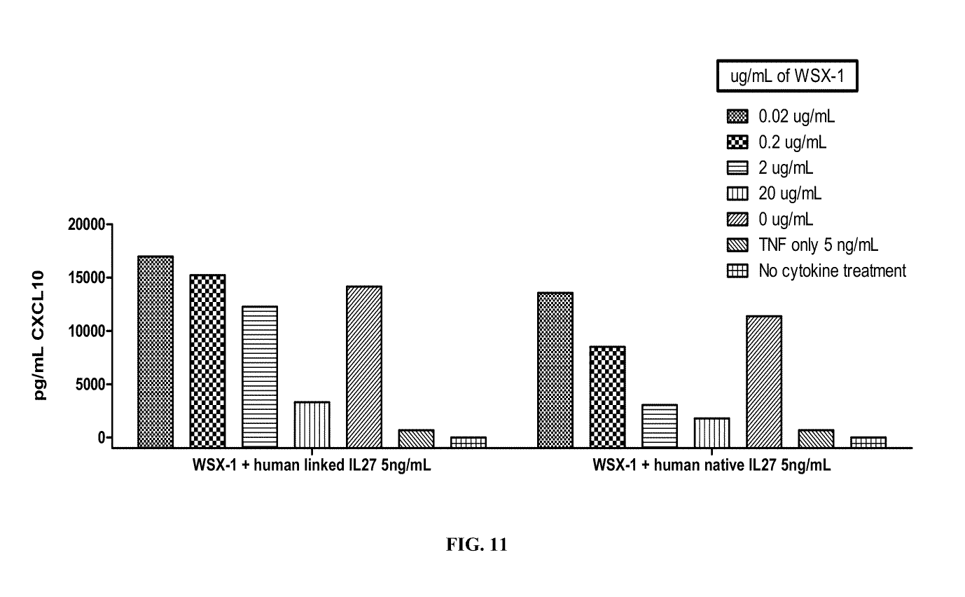

The results of that experiment are shown in FIG. 11. Soluble WSX-1 ECD inhibited IL-27-induced expression of CXCL10.

Various concentrations of a polyclonal antibody against human IL-27 (0.4 to 50 μg/mL; R&D Systems) were preincubated with 10 ng/mL of native human IL-27 in the presence of 5 ng/mL of human TNF-α. Following the preincubation of polyclonal antibody with IL-27 and TNF-α, the stimulus was added to primary human bronchial smooth muscle cells in culture. CXCL10 expression was determined by ELISA.

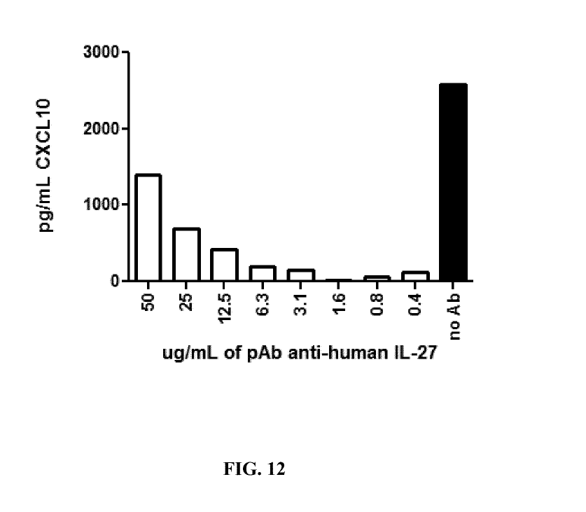

The results of that experiment are shown in FIG. 12. The anti-IL-27 antibody inhibited IL-27-induced expression of CXCL10 in a dose dependent manner.

### TABLE OF SEQUENCES

Table 1 lists certain sequences discussed herein.

TABLE 1
Sequences and Descriptions

| SEQ ID NO | Description | Sequence |
|---|---|---|
| 1 | Human IL-27 subunit | MGQTAGDLGW RLSLLLLPLL LVQAGVWGFP RPPGRPQLSL |
| | p28 | QELRREFTVS LHLARKLLSE VRGQAHRFAE SHLPGVNLYL |
| | | LPLGEQLPDV SLTFQAWRRL SDPERLCFIS TTLQPFHALL |
| | | GGLGTQGRWT NMERMQLWAM RLDLRDLQRH LRFQVLAAGF |
| | | NLPEEEEEEE EEEEEERKGL LPGALGSALQ GPAQVSWPQL |
| | | LSTYRLLHSL ELVLSRAVRE LLLLSKAGHS VWPLGFPTLS |
| | | PQP |
| 2 | Human IL-27 subunit | MTPQLLLALV LWASCPPCSG RKGPPAALTL PRVQCRASRY |
| | EBI3 | PIAVDCSWTL PPAPNSTSPV SFIATYRLGM AARGHSWPCL |
| | | QQTPTSTSCT ITDVQLFSMA PYVLNVTAVH PWGSSSSFVP |
| | | FITEHIIKPD PPEGVRLSPL AERQLQVQWE PPGSWPFPEI |
| | | FSLKYWIRYK RQGAARFHRV GPIEATSFIL RAVRPRARYY |
| | | VQVAAQDLTD YGELSDWSLP ATATMSLGK |
| 3 | Mouse IL-27 subunit | MGQVTGDLGW RLSLLLLPLL LVQAGSWGFP TDPLSLQELR |
| | p28 | REFTVSLYLA RKLLSEVQGY VHSFAESRLP GVNLDLLPLG |
| | | YHLPNVSLTF QAWHHLSDSE RLCFLATTLR PFPAMLGGLG |
| | | TQGTWTSSER EQLWAMRLDL RDLHRHLRFQ VLAAGFKCSK |
| | | EEEDKEEEEE EEEEEKKLPL GALGGPNQVS SQVSWPQLLY |
| | | TYQLLHSLEL VLSRAVRDLL LLSLPRRPGS AWDS |
| 4 | Mouse IL-27 subunit | MSKLLFLSLA LWASRSPGYT ETALVALSQP RVQCHASRYP |
| | EBI3 | VAVDCSWTPL QAPNSTRSTS FIATYRLGVA TQQQSQPCLQ |
| | | RSPQASRCTI PDVHLFSTVP YMLNVTAVHP GGASSSLLAF |
| | | VAERIIKPDP PEGVRLRTAG QRLQVLWHPP ASWPFPDIFS |
| | | LKYRLRYRRR GASHFRQVGP IEATTFTLRN SKPHAKYCIQ |
| | | VSAQDLTDYG KPSDWSLPGQ VESAPHKP |
| 5 | Human IL-27 receptor, | MRGGRGAPFW LWPLPKLALL PLLWVLFQRT RPQGSAGPLQ |
| | alpha subunit (WSX-1), | CYGVGPLGDL NCSWEPLGDL GAPSELHLQS QKYRSNKTQT |
| | with signal peptide | VAVAAGRSWV AIPREQLTMS DKLLVWGTKA GQPLWPPVFV |
| | | NLETQMKPNA PRLGPDVDFS EDDPLEATVH WAPPTWPSHK |
| | | VLICQFHYRR CQEAAWTLLE PELKTIPLTP VEIQDLELAT |
| | | GYKVYGRCRM EKEEDLWGEW SPILSFQTPP SAPKDVWVSG |
| | | NLCGTPGGEE PLLLWKAPGP CVQVSYKVWF WVGGRELSPE |
| | | GITCCCSLIP SGAEWARVSA VNATSWEPLT NLSLVCLDSA |
| | | SAPRSVAVSS IAGSTELLVT WQPGPGEPLE HVVDWARDGD |
| | | PLEKLNWVRL PPGNLSALLP GNFTVGVPYR ITVTAVSASG |
| | | LASASSVWGF REELAPLVGP TLWRLQDAPP GTPAIAWGEV |
| | | PRHQLRGHLT HYTLCAQSGT SPSVCMNVSG NTQSVTLPDL |
| | | PWGPCELWVT ASTIAGQGPP GPILRLHLPD NTLRWKVLPG |
| | | ILFLWGLFLL GCGLSLATSG RCYHLRHKVL PRWVWEKVPD |
| | | PANSSSGQPH MEQVPEAQPL GDLPILEVEE MEPPPVMESS |
| | | QPAQATAPLD SGYEKHFLPT PEELGLLGPP RPQVLA |
| 14 | Human WSX-1, without | QGSAGPLQ CYGVGPLGDL NCSWEPLGDL GAPSELHLQS |
| | signal peptide | QKYRSNKTQT VAVAAGRSWV AIPREQLTMS DKLLVWGTKA |
| | | GQPLWPPVFV NLETQMKPNA PRLGPDVDFS EDDPLEATVH |
| | | WAPPTWPSHK VLICQFHYRR CQEAAWTLLE PELKTIPLTP |
| | | VEIQDLELAT GYKVYGRCRM EKEEDLWGEW SPILSFQTPP |
| | | SAPKDVWVSG NLCGTPGGEE PLLLWKAPGP CVQVSYKVWF |
| | | WVGGRELSPE GITCCCSLIP SGAEWARVSA VNATSWEPLT |
| | | NLSLVCLDSA SAPRSVAVSS IAGSTELLVT WQPGPGEPLE |
| | | HVVDWARDGD PLEKLNWVRL PPGNLSALLP GNFTVGVPYR |
| | | ITVTAVSASG LASASSVWGF REELAPLVGP TLWRLQDAPP |
| | | GTPAIAWGEV PRHQLRGHLT HYTLCAQSGT SPSVCMNVSG |
| | | NTQSVTLPDL PWGPCELWVT ASTIAGQGPP GPILRLHLPD |
| | | NTLRWKVLPG ILFLWGLFLL GCGLSLATSG RCYHLRHKVL |
| | | PRWVWEKVPD PANSSSGQPH MEQVPEAQPL GDLPILEVEE |
| | | MEPPPVMESS QPAQATAPLD SGYEKHFLPT PEELGLLGPP |
| | | RPQVLA |
| 6 | Human gp130, with | MLTLQTWLVQ ALFIFLTTES TGELLDPCGY ISPESPVVQL |
| | signal peptide | HSNFTAVCVL KEKCMDYFHV NANYIVWKTN HFTIPKEQYT |
| | | IINRTASSVT FTDIASLNIQ LICNILTFGQ LEQNVYGITI |
| | | ISGLPPEKPK NLSCIVNEGK KMRCEWDGGR ETHLETNFTL |
| | | KSEWATHKFA DCKAKRDTPT SCTVDYSTVY FVNIEVWVEA |
| | | ENALGKVTSD HINFDPVYKV KPNPPHNLSV INSEELSSIL |
| | | KLTWTNPSIK SVIILKYNIQ YRTKDASTWS QIPPEDTAST |
| | | RSSFTVQDLK PFTEYVFRIR CMKEDGKGYW SDWSEEASGI |
| | | TYEDRPSKAP SFWYKIDPSH TQGYRTVQLV WKTLPPFEAN |
| | | GKILDYEVTL TRWKSHLQNY TVNATKLTVN LTNDRYLATL |
| | | TVRNLVGKSD AAVLTIPACD FQATHPVMDL KAFPKDNMLW |
| | | VEWTTPRESV KKYILEWCVL SDKAPCITDW QQEDGTVHRT |
| | | YLRGNLAESK CYLITVTPVY ADGPGSPESI KAYLKQAPPS |
| | | KGPTVRTKKV GKNEAVLEWD QLPVDVQNGF IRNYTIFYRT |
| | | IIGNETAVNV DSSHTEYTLS SLTSDTLYMV RMAAYTDEGG |
| | | KDGPEFTFTT PKFAQGEIEA IVVPVCLAFL LTTLLGVLFC |
| | | FNKRDLIKKH IWPNVPDPSK SHIAQWSPHT PPRHNFNSKD |
| | | QMYSDGNFTD VSVVEIEAND KKPFPEDLKS LDLFKKEKIN |
| | | TEGHSSGIGG SSCMSSSRPS ISSSDENESS QNTSSTVQYS |
| | | TVVHSGYRHQ VPSVQVFSRS ESTQPLLDSE ERPEDLQLVD |
| | | HVDGGDGILP RQQYFKQNCS QHESSPDISH FERSKQVSSV |
| | | NEEDFVRLKQ QISDHISQSC GSGQMKMFQE VSAADAFGPG |
| | | TEGQVERFET VGMEAATDEG MPKSYLPQTV RQGGYMPQ |
| 18 | Human gp130, without | ELLDPCGY ISPESPVVQL HSNFTAVCVL KEKCMDYFHV |
| | signal peptide | NANYIVWKTN HFTIPKEQYT IINRTASSVT FTDIASLNIQ |
| | | LICNILTFGQ LEQNVYGITI ISGLPPEKPK NLSCIVNEGK |
| | | KMRCEWDGGR ETHLETNFTL KSEWATHKFA DCKAKRDTPT |
| | | SCTVDYSTVY FVNIEVWVEA ENALGKVISD HINFDPVYKV |
| | | KPNPPHNLSV INSEELSSIL KLTWTNPSIK SVIILKYNIQ |
| | | YRTKDASTWS QIPPEDTAST RSSFTVQDLK PFTEYVFRIR |
| | | CMKEDGKGYW SDWSEEASGI TYEDRPSKAP SFWYKIDPSH |
| | | TQGYRTVQLV WKTLPPFEAN GKILDYEVTL TRWKSHLQNY |
| | | TVNATKLTVN LTNDRYLATL TVRNLVGKSD AAVLTIPACD |
| | | FQATHPVMDL KAFPKDNMLW VEWTTPRESV KKYILEWCVL |
| | | SDKAPCITDW QQEDGTVHRT YLRGNLAESK CYLITVTPVY |
| | | ADGPGSPESI KAYLKQAPPS KGPTVRTKKV GKNEAVLEWD |
| | | QLPVDVQNGF IRNYTIFYRT IIGNETAVNV DSSHTEYTLS |
| | | SLTSDTLYMV RMAAYTDEGG KDGPEFTFTT PKFAQGEIEA |
| | | IVVPVCLAFL LTTLLGVLFC FNKRDLIKKH IWPNVPDPSK |
| | | SHIAQWSPHT PPRHNFNSKD QMYSDGNFTD VSVVEIEAND |
| | | KKPFPEDLKS LDLFKKEKIN TEGHSSGIGG SSCMSSSRPS |
| | | ISSSDENESS QNTSSTVQYS TVVHSGYRHQ VPSVQVFSRS |
| | | ESTQPLLDSE ERPEDLQLVD HVDGGDGILP RQQYFKQNCS |
| | | QHESSPDISH FERSKQVSSV NEEDFVRLKQ QISDHISQSC |
| | | GSGQMKMFQE VSAADAFGPG TEGQVERFET VGMEAATDEG |
| | | MPKSYLPQTV RQGGYMPQ |
| 7 | Mouse IL-27 receptor, | MNRLRVARLT PLELLLSLMS LLLGTRPHGS PGPLQCYSVG |
| | alpha subunit (WSX-1), | PLGILNCSWE PLGDLETPPV LYHQSQKYHP NRVWEVKVPS |
| | with signal peptide | KQSWVTIPRE QFTMADKLLI WGTQKGRPLW SSVSVNLETQ |
| | | MKPDTPQIFS QVDISEEATL EATVQWAPPV WPPQKALTCQ |
| | | FRYKECQAEA WTRLEPQLKT DGLTPVEMQN LEPGTCYQVS |
| | | GRCQVENGYP WGEWSSPLSF QTPFLDPEDV WVSGTVCETS |
| | | GKRAALLVWK DPRPCVQVTY TVWFGAGDIT TTQEEVPCCK |
| | | SPVPAWMEWA VVSPGNSTSW VPPTNLSLVC LAPESAPCDV |
| | | GVSSADGSPG IKVTWKQGTR KPLEYVVDWA QDGDSLDKLN |
| | | WTRLPPGNLS TLLPGEFKGG VPYRITVTAV YSGGLAAAPS |
| | | VWGFREELVP LAGPAVWRLP DDPPGTPVVA WGEVPRHQLR |
| | | GQATHYTFCI QSRGLSTVCR NVSSQTQTAT LPNLHSGSFK |
| | | LWVIVSTVAG QGPPGPDLSL HLPDNRIRWK ALPWFLSLWG |
| | | LLLMGCGLSL ASTRCLQARC LHWRHKLLPQ WIWERVPDPA |
| | | NSNSGQPYIK EVSLPQPPKD GPILEVEEVE LQPVVESPKA |
| | | SAPIYSGYEK HFLPTPEELG LLV |
| 15 | Mouse WSX-1, without | TRPHGS PGPLQCYSVG PLGILNCSWE PLGDLETPPV |
| | signal peptide | LYHQSQKYHP NRVWEVKVPS KQSWVTIPRE QFTMADKLLI |
| | | WGTQKGRPLW SSVSVNLETQ MKPDTPQIFS QVDISEEATL |
| | | EATVQWAPPV WPPQKALTCQ FRYKECQAEA WTRLEPQLKT |
| | | DGLTPVEMQN LEPGTCYQVS GRCQVENGYP WGEWSSPLSF |
| | | QTPFLDPEDV WVSGTVCETS GKRAALLVWK DPRPCVQVTY |
| | | TVWFGAGDIT TTQEEVPCCK SPVPAWMEWA VVSPGNSTSW |
| | | VPPTNLSLVC LAPESAPCDV GVSSADGSPG IKVTWKQGTR |
| | | KPLEYVVDWA QDGDSLDKLN WTRLPPGNLS TLLPGEFKGG |
| | | VPYRITVTAV YSGGLAAAPS VWGFREELVP LAGPAVWRLP |
| | | DDPPGTPVVA WGEVPRHQLR GQATHYTFCI QSRGLSTVCR |
| | | NVSSQTQTAT LPNLHSGSFK LWVIVSTVAG QGPPGPDLSL |
| | | HLPDNRIRWK ALPWFLSLWG LLLMGCGLSL ASTRCLQARC |
| | | LHWRHKLLPQ WIWERVPDPA NSNSGQPYIK EVSLPQPPKD |
| | | GPILEVEEVE LQPVVESPKA SAPIYSGYEK HFLPTPEELG |
| | | LLV |
| 8 | Mouse gp130 | MSAPRIWLAQ ALLFFLTTES IGQLLEPCGY IYPEFPVVQR |
| | | GSNFTAICVL KEACLQHYYV NASYIVWKTN HAAVPREQVT |
| | | VINRTTSSVT FTDVVLPSVQ LTCNILSFGQ IEQNVYGVTM |
| | | LSGFPPDKPT NLICIVNEGK NMLCQWDPGR ETYLETNYTL |
| | | KSEWATEKFP DCQSKHGTSC MVSYMPTYYV NIEVWVEAEN |
| | | ALGKVSSESI NFDPVDKVKP TPPYNLSVTN SEELSSILKL |
| | | SWVSSGLGGL LDLKSDIQYR TKDASTWIQV PLEDTMSPRT |
| | | SFTVQDLKPF TEYVFRIRSI KDSGKGYWSD WSEEASGTTY |
| | | EDRPSRPPSF WYKTNPSHGQ EYRSVRLIWK ALPLSEANGK |
| | | ILDYEVILTQ SKSVSQTYTV TGTELTVNLT NDRYVASLAA |
| | | RNKVGKSAAA VLTIPSPHVT AAYSVVNLKA FPKDNLLWVE |
| | | WTPPPKPVSK YILEWCVLSE NAPCVEDWQQ EDATVNRTHL |
| | | RGRLLESKCY QITVTPVFAT GPGGSESLKA YLKQAAPARG |
| | | PTVRIKKVGK NEAVLAWDQI PVDDQNGFIR NYSISYRTSV |
| | | GKEMVVHVDS SHTEYTLSSL SSDTLYMVRM AAYTDEGGKD |
| | | GPEFTFTTPK FAQGEIEAIV VPVCLAFLLT TLLGVLFCFN |
| | | KRDLIKKHIW PNVPDPSKSH IAQWSPHTPP RHNFNSKDQM |
| | | YSDGNFTDVS VVEIEANNKK PCPDDLKSVD LFKKEKVSTE |
| | | GHSSGIGGSS CMSSSRPSIS SNEENESAQS TASTVQYSTV |
| | | VHSGYRHQVP SVQVFSRSES TQPLLDSEER PEDLQLVDSV |
| | | DGGDEILPRQ PYFKQNCSQP EACPEISHFE RSNQVLSGNE |
| | | EDFVRLKQQQ VSDHISQPYG SEQRRLFQEG STADALGTGA |
| | | DGQMERFESV GMETTIDEEI PKSYLPQTVR QGGYMPQ |
| 9 | Human WSX-1 | MRGGRGAPFW LWPLPKLALL PLLWVLFQRT RPQGSAGPLQ |
| | extracellular domain | CYGVGPLGDL NCSWEPLGDL GAPSELHLQS QKYRSNKTQT |
| | (ECD), to aa 482, with | VAVAAGRSWV AIPREQLTMS DKLLVWGTKA GQPLWPPVFV |
| | signal peptide | NLETQMKPNA PRLGPDVDFS EDDPLEATVH WAPPTWPSHK |
| | | VLICQFHYRR CQEAAWTLLE PELKTIPLTP VEIQDLELAT |
| | | GYKVYGRCRM EKEEDLWGEW SPILSFQTPP SAPKDVWVSG |
| | | NLCGTPGGEE PLLLWKAPGP CVQVSYKVWF WVGGRELSPE |
| | | GITCCCSLIP SGAEWARVSA VNATSWEPLT NLSLVCLDSA |
| | | SAPRSVAVSS IAGSTELLVT WQPGPGEPLE HVVDWARDGD |
| | | PLEKLNWVRL PPGNLSALLP GNFTVGVPYR ITVTAVSASG |
| | | LASASSVWGF REELAPLVGP TLWRLQDAPP GTPAIAWGEV |
| | | PRHQLRGHLT HYTLCAQSGT SPSVCMNVSG NTQSVTLPDL |
| | | PW |
| 10 | Human WSX-1 ECD, to | QGSAGPLQ CYGVGPLGDL NCSWEPLGDL GAPSELHLQS |
| | aa 482, without | QKYRSNKTQT VAVAAGRSWV AIPREQLTMS DKLLVWGTKA |
| | signal peptide | GQPLWPPVFV NLETQMKPNA PRLGPDVDFS EDDPLEATVH |
| | | WAPPTWPSHK VLICQFHYRR CQEAAWTLLE PELKTIPLTP |
| | | VEIQDLELAT GYKVYGRCRM EKEEDLWGEW SPILSFQTPP |
| | | SAPKDVWVSG NLCGTPGGEE PLLLWKAPGP CVQVSYKVWF |
| | | WVGGRELSPE GITCCCSLIP SGAEWARVSA VNATSWEPLT |
| | | NLSLVCLDSA SAPRSVAVSS IAGSTELLVT WQPGPGEPLE |
| | | HVVDWARDGD PLEKLNWVRL PPGNLSALLP GNFTVGVPYR |
| | | ITVTAVSASG LASASSVWGF REELAPLVGP TLWRLQDAPP |
| | | GTPAIAWGEV PRHQLRGHLT HYTLCAQSGT SPSVCMNVSG |
| | | NTQSVTLPDL PW |
| 19 | Human WSX-1 ECD, to | MRGGRGAPFW LWPLPKLALL PLLWVLFQRT RPQGSAGPLQ |
| | aa 516, with | CYGVGPLGDL NCSWEPLGDL GAPSELHLQS QKYRSNKTQT |
| | signal peptide | VAVAAGRSWV AIPREQLTMS DKLLVWGTKA GQPLWPPVFV |
| | | NLETQMKPNA PRLGPDVDFS EDDPLEATVH WAPPTWPSHK |
| | | VLICQFHYRR CQEAAWTLLE PELKTIPLTP VEIQDLELAT |
| | | GYKVYGRCRM EKEEDLWGEW SPILSFQTPP SAPKDVWVSG |
| | | NLCGTPGGEE PLLLWKAPGP CVQVSYKVWF WVGGRELSPE |
| | | GITCCCSLIP SGAEWARVSA VNATSWEPLT NLSLVCLDSA |
| | | SAPRSVAVSS IAGSTELLVT WQPGPGEPLE HVVDWARDGD |
| | | PLEKLNWVRL PPGNLSALLP GNFTVGVPYR ITVTAVSASG |
| | | LASASSVWGF REELAPLVGP TLWRLQDAPP GTPAIAWGEV |
| | | PRHQLRGHLT HYTLCAQSGT SPSVCMNVSG NTQSVTLPDL |
| | | PWGPCELWVT ASTIAGQGPP GPILRLHLPD NTLRWK |
| 20 | Human WXS-1 ECD, to | QGSAGPLQCY GVGPLGDLNC SWEPLGDLGA PSELHLQSQK |
| | aa 516, without | YRSNKTQTVA VAAGRSWVAI PREQLTMSDK LLVWGTKAGQ |
| | signal peptide | PLWPPVFVNL ETQMKPNAPR LGPDVDFSED DPLEATVHWA |
| | | PPTWPSHKVL ICQFHYRRCQ EAAWTLLEPE LKTIPLTPVE |
| | | IQDLELATGY KVYGRCRMEK EEDLWGEWSP ILSFQTPPSA |
| | | PKDVWVSGNL CGTPGGEEPL LLWKAPGPCV QVSYKVWFWV |
| | | GGRELSPEGI TCCCSLIPSG AEWARVSAVN ATSWEPLTNL |
| | | SLVCLDSASA PRSVAVSSIA GSTELLVTWQ PGPGEPLEHV |
| | | VDWARDGDPL EKLNWVRLPP GNLSALLPGN FTVGVPYRIT |
| | | VTAVSASGLA SASSVWGFRE ELAPLVGPTL WRLQDAPPGT |
| | | PAIAWGEVPR HQLRGHLTHY TLCAQSGTSP SVCMNVSGNT |
| | | QSVTLPDLPW GPCELWVTAS TIAGQGPPGP ILRLHLPDNT |
| | | LRWK |
| 16 | Mouse WSX-1 | MNRLRVARLT PLELLLSLMS LLLGTRPHGS PGPLQCYSVG |
| | extracellular domain | PLGILNCSWE PLGDLETPPV LYHQSQKYHP NRVWEVKVPS |
| | (ECD), to aa 510, with | KQSWVTIPRE QFTMADKLLI WGTQKGRPLW SSVSVNLETQ |
| | signal peptide | MKPDTPQIFS QVDISEEATL EATVQWAPPV WPPQKALTCQ |
| | | FRYKECQAEA WTRLEPQLKT DGLTPVEMQN LEPGTCYQVS |
| | | GRCQVENGYP WGEWSSPLSF QTPFLDPEDV WVSGTVCETS |
| | | GKRAALLVWK DPRPCVQVTY TVWFGAGDIT TTQEEVPCCK |
| | | SPVPAWMEWA VVSPGNSTSW VPPTNLSLVC LAPESAPCDV |
| | | GVSSADGSPG IKVTWKQGTR KPLEYVVDWA QDGDSLDKLN |
| | | WTRLPPGNLS TLLPGEFKGG VPYRITVTAV YSGGLAAAPS |
| | | VWGFREELVP LAGPAVWRLP DDPPGTPVVA WGEVPRHQLR |
| | | GQATHYTFCI QSRGLSTVCR NVSSQTQTAT LPNLHSGSFK |
| | | LWVIVSTVAG QGPPGPDLSL HLPDNRIRWK |
| 17 | Mouse WSX-1 ECD, to | TRPHGSPGPL QCYSVGPLGI LNCSWEPLGD LETPPVLYHQ |
| | aa 510, without | SQKYHPNRVW EVKVPSKQSW VTIPREQFTM ADKLLIWGTQ |
| | signal peptide | KGRPLWSSVS VNLETQMKPD TPQIFSQVDI SEEATLEATV |
| | | QWAPPVWPPQ KALTCQFRYK ECQAEAWTRL EPQLKIDGLT |
| | | PVEMQNLEPG TCYQVSGRCQ VENGYPWGEW SSPLSFQTPF |
| | | LDPEDVWVSG TVCETSGKRA ALLVWKDPRP CVQVTYTVWF |
| | | GAGDITTTQE EVPCCKSPVP AWMEWAVVSP GNSTSWVPPT |
| | | NLSLVCLAPE SAPCDVGVSS ADGSPGIKVT WKQGTRKPLE |
| | | YVVDWAQDGD SLDKLNWTRL PPGNLSTLLP GEFKGGVPYR |
| | | ITVTAVYSGG LAAAPSVWGF REELVPLAGP AVWRLPDDPP |
| | | GTPVVAWGEV PRHQLRGQAT HYTFCIQSRG LSTVCRNVSS |
| | | QTQTATLPNL HSGSFKLWVT VSTVAGQGPP GPDLSLHLPD |
| | | NRIRWK |
| 11 | Fc C237S | EPKSSDKTHT CPPCPAPELL GGPSVFLFPP KPKDTLMISR |
| | | TPEVTCVVVD VSHEDPEVKF NWYVDGVEVH NAKTKPREEQ |
| | | YNSTYRVVSV LTVLHQDWLN GKEYKCKVSN KALPAPIEKT |
| | | ISKAKGQPRE PQVYTLPPSR DELTKNQVSL TCLVKGFYPS |
| | | DIAVEWESNG QPENNYKTTP PVLDSDGSFF LYSKLTVDKS |
| | | RWQQGNVFSC SVMHEALHNH YTQKSLSLSP GK |
| 12 | Exemplary Fc #1 | ERKCCVECPP CPAPPVAGPS VFLFPPKPKD TLMISRTPEV |
| | | TCVVVDVSHE DPEVQFNWYV DGVEVHNAKT KPREEQFNST |
| | | FRVVSVLTVV HQDWLNGKEY KCKVSNKGLP APIEKTISKT |
| | | KGQPREPQVY TLPPSREEMT KNQVSLTCLV KGFYPSDIAV |
| | | EWESNGQPEN NYKTTPPMLD SDGSFFLYSK LTVDKSRWQQ |
| | | GNVFSCSVMH EALHNHYTQK SLSLSPGK |
| 13 | Exemplary Fc #2 | ESKYGPPCPS CPAPEFLGGP SVFLFPPKPK DTLMISRTPE |
| | | VTCVVVDVSQ EDPEVQFNWY VDGVEVHNAK TKPREEQFNS |
| | | TYRVVSVLTV LHQDWLNGKE YKCKVSNKGL PSSIEKTISK |
| | | AKGQPREPQV YTLPPSQEEM TKNQVSLTCL VKGFYPSDIA |
| | | VEWESNGQPE NNYKTTPPVL DSDGSFFLYS RLTVDKSRWQ |
| | | EGNVFSCSVM HEALHNHYTQ KSLSLSLGK |

1. A method of treating a condition comprising administering an IL-27 antagonist to a subject with the condition, wherein the condition is selected from steroid-resistant asthma, Th2-low asthma, chronic obstructive pulmonary disease (COPD), steroid-resistant systemic lupus erythematosus (SLE), and steroid-resistant inflammatory bowel disease.

2. A method of treating steroid-resistant airway inflammation, comprising administering an IL-27 antagonist to a subject with steroid-resistant airway inflammation.

3. A method of treating airway hyperresponsiveness, comprising administering an IL-27 antagonist to a subject with airway hyperresponsiveness.

4. The method of any one of claims 1 to 3, wherein the subject has a condition selected from steroid-resistant asthma, Th2-low asthma, and COPD.

5. The method of claim 4, wherein the condition has previously been characterized as having an elevated level of at least one protein selected from CXCL9, CXCL10, CXCL11, CD38, and WSX-1 in a subject's bronchial smooth muscle cells.

6. The method of claim 5, wherein the condition has previously been characterized as having an elevated level of at least one protein selected from CXCL9, CXCL10, CD38, and WSX-1 in a subject's bronchial smooth muscle cells.

7. The method of claim 4, wherein the condition has previously been characterized as having an elevated level of at least one protein selected from WSX-1, CXCL9, CXCL10, and CXCL11 in a subject's bronchial epithelial cells.

8. The method of claim 7, wherein the condition has previously been characterized as having an elevated level of at least one protein selected from CXCL9 and CXCL10 in a subject's bronchial epithelial cells.

9. A method of reducing expression of at least one gene selected from CXCL10, CXCL9, CXCL11, CD38, and WSX-1 in bronchial smooth muscle cells or bronchial epithelial cells comprising contacting the cells with an IL-27 antagonist.

10. A method of increasing the steroid sensitivity of bronchial smooth muscle cells or bronchial epithelial cells comprising contacting the cells with an IL-27 antagonist.

11. The method of any one of the preceding claims, wherein the IL-27 antagonist is selected from an antibody that binds IL-27, an antibody that binds p28, an antibody that binds EBI3, an antibody that binds IL-27 receptor (IL-27R), an antibody that binds WSX-1, a WSX-1 extracellular domain (ECD), and a WSX-1 ECD fusion molecule.

12. The method of claim 11, wherein the IL-27 antagonist is selected from an antibody that binds IL-27, an antibody that binds p28, and an antibody that binds EBI3.

13. The method of claim 12, wherein the IL-27 antagonist is an antibody that binds p28.

14. The method of claim 13, wherein the antibody binds p28, but does not bind to EBI3.

15. The method of claim 14, wherein the antibody binds to the IL-27 heterodimer.

16. The method of any one of claims 12 to 15, wherein the antibody is selected from a chimeric antibody, a humanized antibody, and a human antibody.

17. The method of any one of claims 12 to 16, wherein the antibody is an antibody fragment.

18. The method of claim 17, wherein the antibody fragment is selected from an Fv, a single-chain Fv (scFv), a Fab, a Fab′, and a (Fab′)₂.

19. The method of any one of claims 1 to 8, further comprising administering the subject at least one additional therapeutic selected from an anti-inflammatory agent and a bronchodilator.

20. The method of claim 19, wherein the additional therapeutic is an anti-inflammatory agent.

21. The method of claim 20, wherein the anti-inflammatory agent is selected from a steroid, a mast cell stabilizer, a leukotriene antagonist, omalizumab, roflumilast, and cilomilast.

22. The method of claim 21, wherein the steroid is selected from prednisone, prednisolone, methylprednisone, fluticasone, budesonide, mometasone, triamcinolone, beclometasone, dexamethasone, and betamethasone; the mast cell stabilizer is selected from cromoglicic acid, nedocromil sodium; and the leukotriene antagonist is selected from montelukast, zafirlukast, and zileuton.

23. The method of claim 19, wherein the additional therapeutic is a bronchodilator.

24. The method of claim 23, wherein the bronchodilator is selected from β₂ agonist, an anticholinergic, and theophylline.

25. The method of claim 24, wherein the β₂ agonist is selected from albuterol, terbutaline, slameterol, and formoterol; and the anticholinergic is selected from ipratropium and tiotropium.
